(19) United States
(12) Patent Application Publication
(10) Pub. No.: US 2008/0058747 A1
(43) Pub. Date: Mar. 6, 2008

(54) Absorbent articles comprising superabsorbent polymers having superior properties

(76) Inventors: Arvinder Pal Singh Kainth, Menasha, WI (US); Jason Matthew Laumer, Appleton, WI (US); William G.-J. Chiang, Yorktown, VA (US); Norbert Herfert, Altenstadt (DE); Michael A. Mitchell, Waxhaw, NC (US); Richard Keith Goodwin, Suffolk, VA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/514,540

(22) Filed: Aug. 31, 2006

(51) Int. Cl. A61F 13/15

(52) U.S. Cl. 604/368; 604/372

(57) ABSTRACT

An absorbent article can have a topsheet, a backsheet, and an absorbent core disposed between the topsheet and the backsheet. In some aspects, at least one of the topsheet, backsheet, and absorbent core is stretchable. In other aspects, the absorbent core can comprise layers, at least one of which includes substantially the superabsorbent material and at least one of which includes substantially fluff. In some aspects, the article includes superabsorbent material which has a centrifuge retention capacity of at least about 25 g/g, a free swell gel bed permeability of at least 200 Darcies, and a gel integrity of at least 2. In some aspects, the superabsorbent material is coated with a polyvinylamine.

### BACKGROUND

Articles, such as absorbent articles, are useful for absorbing many types of fluids, including fluids secreted or eliminated by the human body. Superabsorbent polymers (SAPs) are frequently used in absorbent articles to help improve the absorbent properties of such articles. SAPs are generally polymer based and are available in many forms, such as powders, granules, microparticles, films and fibers, for example. Upon contact with fluids, such SAPs swell by absorbing the fluids into their structures. In general, SAPs can quickly absorb fluids insulted into such articles, and can retain such fluids to prevent leakage and help provide a dry feel even after fluid insult.

There is a continuing effort to improve the performance of such absorbent articles, especially at high levels of fluid saturation, to thereby reduce the occurrence of leakage and to improve fit and comfort. This is particularly significant when such articles are subjected to repeated fluid insults during use. This has become an increasing challenge as recent efforts in absorbent article design have generally focused on using higher concentrations of superabsorbent material and less fluff fibers to make the absorbent structures thinner and more flexible. However, notwithstanding the increase in total absorbent capacity obtained by increasing the concentration of superabsorbent material, such absorbent articles may still nevertheless leak during use. Such leakage may in part be the result of the absorbent core component of an article having an insufficient intake rate (i.e., the rate at which a fluid insult can be taken into and entrained within the absorbent core for subsequent absorption by the superabsorbent material) due to low permeability and lack of available void volume. Therefore, there is a desire for an absorbent article which contains high levels of superabsorbent materials and which can maintain a sufficient intake rate.

In addition, there is also a need for superabsorbent polymer materials that have increased permeability characteristics while retaining other characteristics such as adequate absorption and retention. Permeability is a measure of the effective connectedness of a porous structure, be it a mat of fiber or a slab of foam or, in this case, crosslinked polymers and may be specified in terms of the void fraction and extent of connectedness of the superabsorbent polymer material. Gel permeability is a property of the mass of particles as a whole and is related to particle size distribution, particle shape, and the connectedness of the open pores, shear modulus and surface modification of the swollen gel. In practical terms, the permeability of the superabsorbent polymer material is a measure of how rapidly liquid flows through the mass of swollen particles. Low permeability indicates that liquid cannot flow readily through the superabsorbent polymer material, which is generally referred to as gel blocking, and that any forced flow of liquid (such as a second application of urine during use of the diaper) must take an alternate path (e.g., diaper leakage). Therefore, there is a desire for an absorbent article which exhibits increased permeability characteristics.

One desirable method of improving the absorption and retention properties of SAP particles is to surface treat the SAP particles. The surface treatment of SAP particles with crosslinking agents having two or more functional groups capable of reacting with pendant carboxylate groups on the polymer comprising the SAP particle is disclosed in numerous patents. Surface treatment improves absorbency and gel rigidity to increase fluid flowability and prevent SAP particle agglomeration, and improves gel strength.

Surface-crosslinked SAP particles, in general, exhibit higher liquid absorption and retention values than SAP particles having a comparable level of internal crosslinks, but lacking surface crosslinks. Internal crosslinks arise from polymerization of the monomers comprising the SAP particles, and are present in the polymer backbone. It has been theorized that surface crosslinking increases the resistance of SAP particles to deformation, thus reducing the degree of contact between surfaces of neighboring SAP particles when the resulting hydrogel is deformed under an external pressure. The degree to which absorption and retention values are enhanced by surface crosslinking is related to the relative amount and distribution of internal and surface crosslinks, and to the particular surface crosslinking agent and method of surface crosslinking.

### SUMMARY

In response to the needs discussed above, an article of the present invention comprises an absorbent article which can have a topsheet, a backsheet, and an absorbent core disposed between the topsheet and the backsheet. The absorbent core includes superabsorbent polymer particles (SAP particles) having a centrifuge retention capacity of at least about 25 g/g, a free swell gel bed permeability of at least 200 Darcies, and a gel integrity of at least 2. In some aspects, at least one of the topsheet, backsheet, and absorbent core is stretchable. In other aspects, the absorbent core can comprise layers, at least one of which includes substantially the superabsorbent material and at least one of which includes substantially fluff.

In some aspects, the superabsorbent polymer particles have a free swell gel bed permeability of at least 250 Darcies. In other aspects, the superabsorbent polymer particles have a gel integrity of at least 2.5. In still other aspects, the surfaces of the superabsorbent polymer particles are hydrophobic, while in other aspects, the surfaces of the superabsorbent polymer particles are hydrophilic.

In some aspects, the superabsorbent polymer particles have been prepared by a method where particles of a surface-crosslinked superabsorbent polymer are coated with a coating composition including a polyamine and water; and the polyamine-coated polymer particles are maintained at 25° C. to 100° C. for about 5 to about 60 minutes. In further aspects, these superabsorbent polymer particles have a hydrophilic surface. In other aspects, the surface-crosslinked superabsorbent polymer comprises acrylic acid, methacrylic acid, or a mixture thereof. In yet other aspects, the surface-crosslinked superabsorbent polymer has a degree of neutralization of about 25 to about 100.

In some aspects, the polyamine is present on surfaces of the surface-crosslinked superabsorbent polymer in an amount of about 0.1% to about 2%, by weight, of the particle. In other aspects, the polyamine has one or more of primary amino groups, secondary amino groups, tertiary amino groups, and quaternary ammonium groups. In still other aspects, the polyamine has a weight average molecular weight of about 5,000 to about 1,000,000. In yet other aspects, the polyamine is a homopolymer or a copolymer selected from the group consisting of a polyvinylamine, a polyethyleneimine, a polyallylamine, a polyalkyleneamine, a polyazetidine, a polyvinylguanidine, a poly(DADMAC), a cationic polyacrylamide, a polyamine functionalized polyacrylate, and mixtures thereof.

In some aspects, the surface-crosslinked superabsorbent polymer further comprises a crosslinking agent. In other aspects, the crosslinking agent comprises a salt having (a) a polyvalent metal cation of valence +2, +3, or +4, (b) a polyvalent anion of valence 2, 3, or 4, or (c) a polyvalent cation and a polyvalent anion. In further aspects, the polyvalent metal cation is selected from the group consisting of Mg2+, Ca2+, Al3+, Sc3+, Ti4+, Mn2+, Fe2+/3+, CO2+, Ni2+, Cu+/2+, Zn2+, Y3+, Zr4+, La3+, Ce4+, Hf4+, Au3+, and mixtures thereof. In other aspects, the polyvalent anion is selected from the group consisting of sulfate, phosphate, hydrogen phosphate, borate, an anion of a polycarboxylic acid, and mixtures thereof. In still other aspects, the crosslinking agent comprises a multifunctional organic component capable of reacting with amino groups of the polyamine. In yet other aspects, the crosslinking agent is selected from an alkylene carbonate, a polyaziridine, a haloepoxy, a polyisocyanate, a di or polyglycidyl compound, a alkoxysilyl compound, urea, thiourea, guanidine, dicyandiamide, 2-oxazolidinone or a derivative thereof, bisoxazoline, a polyoxazoline, di and polyisocyanate, di and poly-N-methylol compounds, or a compound having two or more blocked isocyanate groups, a multifunctional aldehyde, a multifunctional ketone, a multifunctional acetal, a multifunctional ketal, or mixtures thereof.

In some aspects, the surface-crosslinked superabsorbent polymer coating composition comprises a cosolvent. In further aspects, these superabsorbent polymer particles have a hydrophobic surface. In other aspects, the cosolvent comprises an alcohol, a diol, a triol, or a mixture thereof. In still other aspects, the cosolvent comprises methanol, ethanol, propyl alcohol, isopropyl alcohol, ethylene glycol, propylene glycol, an oligomer of ethylene glycol, an oligomer of propylene glycol, glycerin, a monoalkyl ether of propylene glycol, and mixtures thereof.

In some aspects of the invention, the surface-crosslinked superabsorbent polymer comprises a surface-crosslinked polyacrylic acid. In other aspects of the invention, the polyamine comprises a polyvinylamine homopolymer or copolymer.

In some aspects, an absorbent article can have a topsheet, a backsheet, and an absorbent core disposed between the topsheet and the backsheet, and can include superabsorbent polymer particles that have been prepared by: (a) providing surface-crosslinked superabsorbent polymer particles; (b) applying a composition comprising a polyamine, an optional cosolvent, and an optional crosslinking agent to surfaces of the surface-crosslinked polymer particles; and (c) maintaining the coated surface-crosslinked polymer particles of step (b) at about 25° C. to about 100° C. for a sufficient time to provide a cured polyamine coating on the surface-crosslinked polymer particles. In further aspects, maintaining step (c) is performed at about 50° C. to about 100° C. for about 5 minutes to about 60 minutes.

In some aspects, an absorbent article comprises a topsheet, a backsheet, and an absorbent core disposed between the topsheet and the backsheet; where the absorbent core includes polyamine-coated superabsorbent polymer particles having a hydrophobic surface; and where the polyamine-coated superabsorbent polymer particles have a wicking index of less than 2.3 centimeters after one minute, a free swell gel bed permeability at least two times greater than identical superabsorbent polymer particles free of a polyamine coating, and a gel integrity of at least 2.5. In some aspects, at least one of the topsheet, backsheet, and absorbent core is stretchable. In other aspects, the absorbent core can comprise layers, at least one of which includes substantially the superabsorbent material and at least one of which includes substantially fluff.

In some aspects, the polyamine-coated superabsorbent polymer particles have a wicking index of less than 3 centimeters after 5 minutes. In other aspects, the polyamine-coated superabsorbent polymer particles have a wicking index of less than 6.5 centimeters after 10 minutes. In still other aspects, the polyamine-coated superabsorbent polymer particles have a gel integrity of at least 3.0. In yet other aspects, the polyamine-coated superabsorbent polymer particles have a gel integrity of at least 3.5. In still other aspects, the polyamine-coated superabsorbent polymer particles, after contact with a 0.9% saline solution, exhibit a delay of at least five seconds prior to absorbing the saline solution.

In some aspects, the polyamine-coated superabsorbent polymer particles have been prepared by a method where particles of a surface-crosslinked superabsorbent polymer are coated with a coating composition comprising a polyamine, a cosolvent, and water; and the polyamine-coated polymer particles are maintained at about 25° C. to about 100° C. for about 5 to about 60 minutes.

In some aspects, the crosslinked superabsorbent polymer comprises acrylic acid, methacrylic acid, or a mixture thereof. In other aspects, the crosslinked superabsorbent polymer has a degree of neutralization of about 25 to about 100.

In some aspects, the polyamine is present on surfaces of the surface-crosslinked superabsorbent polymer in an amount of about 0.1% to about 2%, by weight, of the particle. In other aspects, the polyamine has one or more of primary amino groups, secondary amino groups, tertiary amino groups, and quaternary ammonium groups. In still other aspects, the polyamine has a weight average molecular weight of about 5,000 to about 1,000,000. In yet other aspects, the polyamine is a homopolymer or a copolymer selected from the group consisting of a polyvinylamine, a polyethyleneimine, a polyallylamine, a polyalkyleneamine, a polyazetidine, a polyvinylguanidine, a poly(DADMAC), a cationic polyacrylamide, a polyamine functionalized polyacrylate, and mixtures thereof. In yet other aspects, the polyamine comprises a polyvinylamine homopolymer or copolymer.

In some aspects, the cosolvent comprises an alcohol, a diol, a triol, or a mixture thereof. In other aspects, the cosolvent comprises methanol, ethanol, propyl alcohol, isopropyl alcohol, ethylene glycol, propylene glycol, an oligomer of ethylene glycol, an oligomer of propylene glycol, glycerin, a monoalkyl ether of propylene glycol, and mixtures thereof.

In some aspects, the surface-crosslinked superabsorbent polymer comprises a surface-crosslinked polyacrylic acid.

In some aspects, an absorbent article can have a topsheet, a backsheet, and an absorbent core disposed between the topsheet and the backsheet, and can include superabsorbent polymer particles that have been prepared by: (a) providing surface-crosslinked superabsorbent polymer particles; (b) applying a composition comprising a polyamine and a cosolvent to surfaces of the surface-crosslinked polymer particles; and (c) maintaining the coated surface-crosslinked polymer particles of step (b) at about 25° C. to about 100° C. for a sufficient time to provide a cured, hydrophobic polyamine coating on the surface-crosslinked polymer particles. In further aspects, maintaining step (c) is performed at about 50° C. to about 100° C. for about 5 minutes to about 60 minutes. In other aspects, step (b) and step (c) are performed simultaneously.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1 is a partially cut away top view of a Saturated Capacity tester;

FIG. 2 is a side view of a Saturated Capacity tester;

FIG. 3 is a rear view of a Saturated Capacity tester;

FIG. 4 is a top view of the test apparatus employed for the Fluid Intake Rate Test;

FIG. 5 is a side view of the test apparatus employed for the Fluid Intake Rate Test;

FIG. 6 is a perspective view of one embodiment of an absorbent article that may be made in accordance with the present invention;

FIG. 7 is a plan view of the absorbent article shown in FIG. 6 with the article in an unfastened, unfolded and laid flat condition showing the surface of the article that faces the wearer when worn and with portions cut away to show underlying features;

FIG. 8 is a schematic diagram of one version of a method and apparatus for producing an absorbent core;

FIG. 9 is a cross-sectional side view of a layered absorbent core according to the present invention;

FIG. 10A is a cross-section side view of an absorbent bandage of the present invention;

FIG. 10B is a top perspective view of an absorbent bandage of the present invention;

FIG. 11 is a top perspective view of an absorbent bed or furniture liner of the present invention;

FIG. 12 is a perspective view of an absorbent sweatband of the present invention;

FIGS. 13-15 contain a sequence of photographs showing the addition of 0.9% saline to SAP particles and the subsequent swelling of the wetted SAP particles over the first 30 seconds after the saline addition.

Repeated use of reference characters in the present specification and drawings is intended to represent the same or analogous features or elements of the present invention.

### Test Methods

### Centrifuge Retention Capacity Test (CRC)

This test determines the free swelling capacity of a hydrogel-forming polymer. The resultant retention capacity is stated as grams of liquid retained per gram weight of the sample (g/g). In this method, 0.2000±0.0050 g of dry SAP particles of size fraction 106 to 850 μm are inserted into a teabag. A heat-sealable tea bag material, such as that available from Dexter Corporation (having a place of business in Windsor Locks, Conn., U.S.A.) as model designation 1234T heat sealable filter paper works well for most applications. The bag is formed by folding a 5-inch by 3-inch sample of the bag material in half and heat-sealing two of the open edges to form a 2.5-inch by 3-inch rectangular pouch. The heat seals should be about 0.25 inches inside the edge of the material. After the sample is placed in the pouch, the remaining open edge of the pouch is also heat-sealed. Empty bags can also be made to serve as controls. The teabag is placed in saline solution (i.e., 0.9 wt % aqueous sodium chloride) for 30 minutes (at least 0.83 1 (liter) saline solution/1 g polymer), making sure that the bags are held down until they are completely wetted. Then, the teabag is centrifuged for 3 minutes at 250 G. The absorbed quantity of saline solution is determined by measuring the weight of the teabag. The amount of solution retained by the superabsorbent polymer sample, taking into account the solution retained by the bag itself, is the centrifuge retention capacity (CRC) of the superabsorbent polymer, expressed as grams of fluid per gram of superabsorbent polymer. More particularly, the retention capacity is determined by the following equation:

$$\frac{\begin{matrix}sample \text{/} bag after centrifuge ] - [ empty \\ bag after centrifuge ] - [ dry sample weight ]\end{matrix}}{[ dry sample weight ]}$$

### Free-Swell Gel Bed Permeability Test (FSGBD)

This procedure is disclosed in U.S. Patent Publication No. 2005/0256757 to Sierra et al., incorporated herein by reference in a manner that is consistent herewith. The results are expressed in Darcies.

### Gel Bed Permeability Test (GBP 0.3 psi)

This procedure is disclosed in U.S. Patent Publication No. 2005/0256757 to Sierra et al., incorporated herein by reference in a manner that is consistent herewith, except the method is modified by using a 100 gram weight to provide 0.3 psi. The results are expressed in Darcies.

### Absorbency Under Load Test (AUL)

This procedure is disclosed in WO 00/62825 to Reeves et al., pages 22-23, incorporated herein by reference in a manner that is consistent herewith, using a 317 gram weight for an AUL (0.90 psi).

### Gel Integrity (GI)

In a small polystyrene weighing dish (one inch diameter base) place a 1 g of an SAP sample. The SAP particles are evenly distributed on the bottom of the dish, then 1 g of 0.9 wt % saline is added to the center of the SAP particles. The particles are allowed to stand for one minute and then evaluated:

| Properties | Grade |
|---|---|
| Loose particles, unable to pick up as one mass. | 1 |
| Can lift as one mass with thumb and forefinger, but tears under | 2 |
| its own weight. | |
| Can lift as one mass with thumb and forefinger, but tears when | 3 |
| oscillated in the horizontal direction. | |
| Can lift as one mass with thumb and forefinger, but tears with | 4 |
| the use of the opposing thumb and forefinger. | |

### Fluid Wicking Index

This procedure is identical to that disclosed in European Patent No. EP 0 532 002 B1 to Byerly et al., incorporated herein by reference in a manner that is consistent herewith.

### Particle Size Distribution (PSD)

A sample of SAP particles is added to the top of a series of stacked sieves, each of which has consecutively smaller openings. The sieves are mechanically shaken for a predetermined time, then the amount of SAP particles on each sieve is weighed. The percent of superabsorbent material on each sieve is calculated from the initial sample weight of the SAP particles sample.

### Saturated Capacity Test

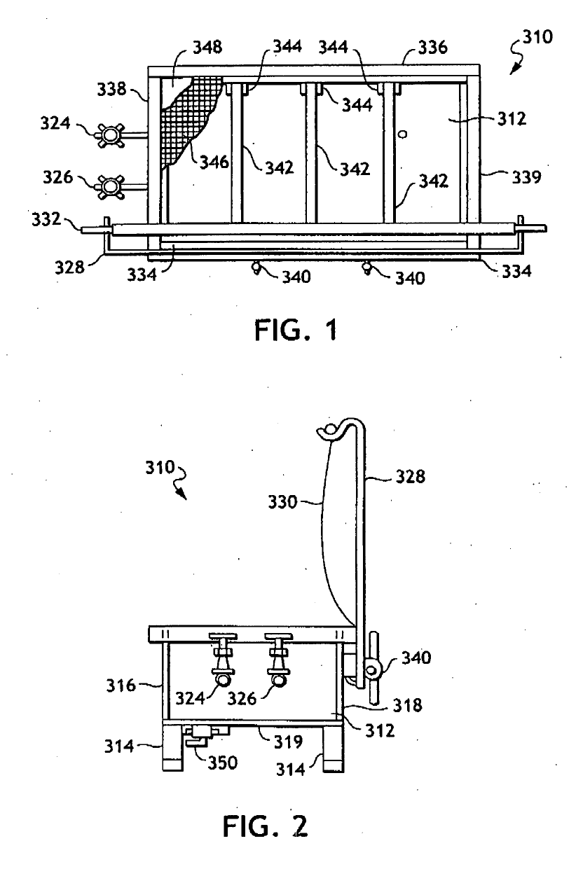

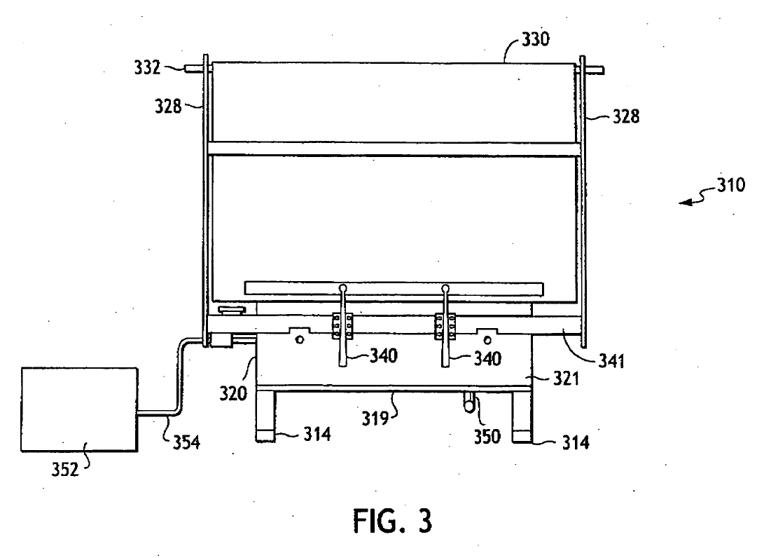

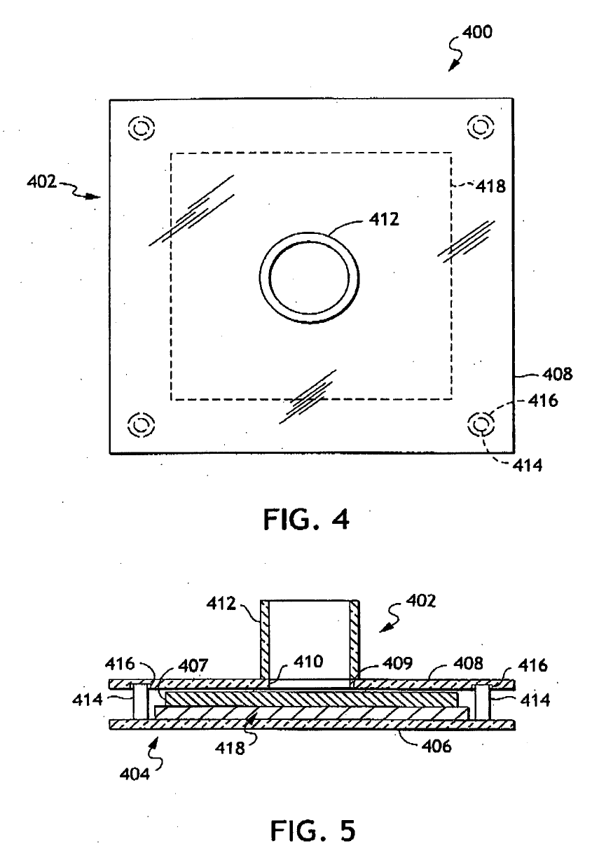

Saturated Capacity is determined using a Saturated Capacity (SAT CAP) tester with a Magnahelic vacuum gage and a latex dam, comparable to the following description. Referring to FIGS. 1-3, a Saturated Capacity tester vacuum apparatus 310 comprises a vacuum chamber 312 supported on four leg members 314. The vacuum chamber 312 includes a front wall member 316, a rear wall member 318, and two side walls 320 and 321. The wall members are sufficiently thick to withstand the anticipated vacuum pressures, and are constructed and arranged to provide a chamber having outside dimensions measuring 23.5 inches (59.7 cm) in length, 14 inches (35.6 cm) in width and 8 inches (20.3 cm) in depth.

A vacuum pump (not shown) operably connects with the vacuum chamber 312 through an appropriate vacuum line conduit and a vacuum valve 324. In addition, a suitable air bleed line connects into the vacuum chamber 312 through an air bleed valve 326. A hanger assembly 328 is suitably mounted on the rear wall 318 and is configured with S-curved ends to provide a convenient resting place for supporting a latex dam sheet 330 in a convenient position away from the top of the vacuum apparatus 310. A suitable hanger assembly can be constructed from 0.25 inch (0.64 cm) diameter stainless steel rod. The latex dam sheet 330 is looped around a dowel member 332 to facilitate grasping and to allow a convenient movement and positioning of the latex dam sheet 330. In the illustrated position, the dowel member 332 is shown supported in a hanger assembly 328 to position the latex dam sheet 330 in an open position away from the top of the vacuum chamber 312.

A bottom edge of the latex dam sheet 330 is clamped against a rear edge support member 334 with suitable securing means, such as toggle clamps 340. The toggle clamps 340 are mounted on the rear wall member 318 with suitable spacers 341 which provide an appropriate orientation and alignment of the toggle clamps 340 for the desired operation. Three support shafts 342 are 0.75 inches in diameter and are removably mounted within the vacuum chamber 312 by means of support brackets 344. The support brackets 344 are generally equally spaced along the front wall member 316 and the rear wall member 318 and arranged in cooperating pairs. In addition, the support brackets 344 are constructed and arranged to suitably position the uppermost portions of the support shafts 342 flush with the top of the front, rear and side wall members of the vacuum chamber 312. Thus, the support shafts 342 are positioned substantially parallel with one another and are generally aligned with the side wall members 320 and 321. In addition to the rear edge support member 334, the vacuum apparatus 310 includes a front support member 336 and two side support members 338 and 339. Each side support member measures about 1 inch (2.5 cm) in width and about 1.25 inches (3.2 cm) in height. The lengths of the support members are constructed to suitably surround the periphery of the open top edges of the vacuum chamber 312, and are positioned to protrude above the top edges of the chamber wall members by a distance of about 0.5 inches.

A layer of egg crating type material 346 is positioned on top of the support shafts 342 and the top edges of the wall members of the vacuum chamber 312. The egg crate material extends over a generally rectangular area measuring 23.5 inches (59.7 cm) by 14 inches (35.6 cm), and has a depth measurement of about 0.38 inches (1.0 cm). The individual cells of the egg crating structure measure about 0.5 inch square, and the thin sheet material comprising the egg crating is composed of a suitable material, such as polystyrene. For example, the egg crating material can be McMaster-Carr Supply Catalog No. 162 4K 14 (available from McMaster-Carr Supply Company, having a place of business in Atlanta, Ga. U.S.A.) translucent diffuser panel material. A layer of 6 mm (0.24 inch) mesh TEFLON-coated screening 348 (available from Eagle Supply and Plastics, Inc., having a place of business in Appleton, Wis., U.S.A.) which measures 23.5 inches (59.7 cm) by 14 inches (35.6 cm), is placed on top of the egg crating material 346.

A suitable drain line and a drain valve 350 connect to the bottom plate member 319 of the vacuum chamber 312 to provide a convenient mechanism for draining liquids from the vacuum chamber 312. The various wall members and support members of the vacuum apparatus 310 may be composed of a suitable non-corroding, moisture-resistant material, such as polycarbonate plastic. The various assembly joints may be affixed by solvent welding and/or fasteners, and the finished assembly of the tester is constructed to be water-tight. A vacuum gauge 352 operably connects through a conduit into the vacuum chamber 312. A suitable pressure gauge is a Magnahelic differential gauge capable of measuring a vacuum of 0-100 inches of water, such as a No. 2100 gauge available from Dwyer Instrument Incorporated (having a place of business in Michigan City, Ind., U.S.A.)

The dry product or other absorbent structure is weighed and then placed in excess 0.9% NaCl saline solution, submerged and allowed to soak for twenty (20) minutes. After the twenty (20) minute soak time, the absorbent structure is placed on the egg crate material and mesh TEFLON-coated screening of the Saturated Capacity tester vacuum apparatus 310. The latex dam sheet 330 is placed over the absorbent structure(s) and the entire egg crate grid so that the latex dam sheet 330 creates a seal when a vacuum is drawn on the vacuum apparatus 310. A vacuum of 0.5 pounds per square inch (psi) is held in the Saturated Capacity tester vacuum apparatus 310 for five minutes. The vacuum creates a pressure on the absorbent structure(s), causing drainage of some liquid. After five minutes at 0.5 psi vacuum, the latex dam sheet 330 is rolled back and the absorbent structure(s) are weighed to generate a wet weight.

The overall capacity of each absorbent structure is determined by subtracting the dry weight of each absorbent from the wet weight of that absorbent, determined at this point in the procedure. The 0.5 psi Saturated Capacity or Saturated Capacity of the absorbent structure is determined by the following formula:

Saturated Capacity=(wet weight−dry weight)/dry weight;

wherein the Saturated Capacity value has units of grams of fluid/gram of absorbent. For Saturated Capacity, a minimum of three specimens of each sample should be tested and the results averaged. If the absorbent structure has low integrity or disintegrates during the soak or transfer procedures, the absorbent structure can be wrapped in a containment material such as paper toweling, for example SCOTT paper towels manufactured by Kimberly-Clark Corporation, having a place of business in Neenah, Wis., U.S.A. The absorbent structure can be tested with the overwrap in place and the capacity of the overwrap can be independently determined and subtracted from the wet weight of the total wrapped absorbent structure to obtain the wet absorbent weight.

### Fluid Intake Rate Test

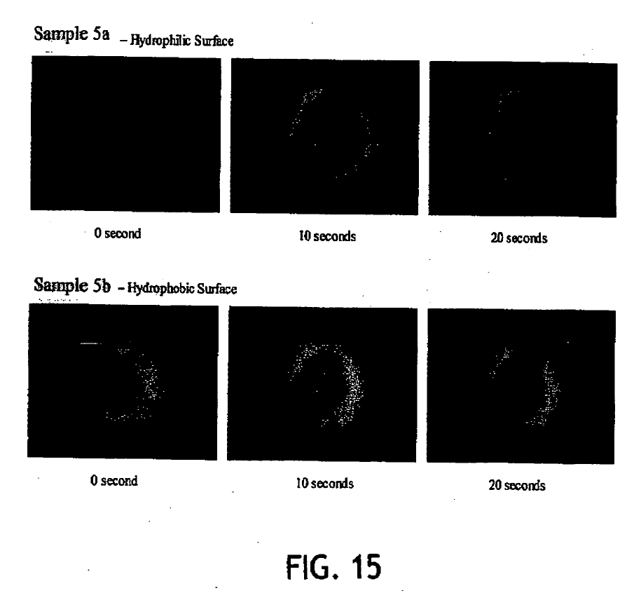

The Fluid Intake Rate (FIR) Test determines the amount of time required for an absorbent structure to take in (but not necessarily absorb) a known amount of test solution (0.9 weight percent solution of sodium chloride in distilled water at room temperature). A suitable apparatus for performing the FIR Test is shown in FIGS. 15 and 16 and is generally indicated at 400. The test apparatus 400 comprises upper and lower assemblies, generally indicated at 402 and 404 respectively, wherein the lower assembly comprises a generally 7 inch by 7 inch (17.8 cm×17.8 cm) square lower plate 406 constructed of a transparent material such as PLEXIGLAS (available from Degussa AG, having a place of business in Dusseldorf, Germany) for supporting the absorbent sample during the test and a generally 4.5 inch by 4.5 inch (11.4 cm×11.4 cm) square platform 418 centered on the lower plate 406.

The upper assembly 402 comprises a generally square upper plate 408 constructed similar to the lower plate 406 and having a central opening 410 formed therein. A cylinder (fluid delivery tube) 412 having an inner diameter of about one inch (2.5 cm) is secured to the upper plate 408 at the central opening 410 and extends upward substantially perpendicular to the upper plate. The central opening 410 of the upper plate 408 should have a diameter at least equal to the inner diameter of the cylinder 412 where the cylinder 412 is mounted on top of the upper plate 408. However, the diameter of the central opening 410 may instead be sized large enough to receive the outer diameter of the cylinder 412 within the opening so that the cylinder 412 is secured to the upper plate 408 within the central opening 410.

Pin elements 414 are located near the outside corners of the lower plate 406, and corresponding recesses 416 in the upper plate 408 are sized to receive the pin elements 414 to properly align and position the upper assembly 402 on the lower assembly 404 during testing. The weight of the upper assembly 402 (e.g., the upper plate 408 and cylinder 412) is approximately 360 grams to simulate approximately 0.11 pounds/square inch (psi) pressure on the absorbent sample during the FIR Test.

To run the FIR Test, an absorbent sample 407 being three inches (7.6 cm) in diameter is weighed and the weight is recorded in grams. The sample 407 is then centered on the platform 418 of the lower assembly 404. The upper assembly 402 is placed over the sample 407 in opposed relationship with the lower assembly 404, with the pin elements 414 of the lower plate 406 seated in the recesses 416 formed in the upper plate 408 and the cylinder 412 is generally centered over the sample 407. Prior to running the FIR Test, the aforementioned Saturated Capacity Test is measured on the sample 407. Thirty percent (30%) of the saturation capacity is then calculated by multiplying the mass of the dry sample (grams) times the measured saturated capacity (gram/gram) times 0.3; e.g., if the test sample has a saturated capacity of 20 g of 0.9% NaCl saline test solution/g of test sample and the three inch (7.6 cm) diameter sample 407 weighs one gram, then 6 grams of 0.9% NaCl saline test solution (referred to herein as a first insult) is poured into the top of the cylinder 412 and allowed to flow down into the absorbent sample 407. A stopwatch is started when the first drop of solution contacts the sample 407 and is stopped when the liquid ring between the edge of the cylinder 412 and the sample 407 disappears. The reading on the stopwatch is recorded to two decimal places and represents the intake time (in seconds) required for the first insult to be taken into the absorbent sample 407.

A time period of fifteen minutes is allowed to elapse, after which a second insult equal to the first insult is poured into the top of the cylinder 412 and again the intake time is measured as described above. After fifteen minutes, the procedure is repeated for a third insult. An intake rate (in milliliters/second) for each of the three insults is determined by dividing the amount of solution (e.g., six grams) used for each insult by the intake time measured for the corresponding insult.

At least three samples of each absorbent test are subjected to the FIR Test and the results are averaged to determine the intake rate.

### Definitions

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to devices which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, household/industrial absorbent articles, and sports/construction absorbent articles.

As used herein, the terms “base polymer particles,” “surface-crosslinked SAP particles,” and “SAP particles” refer to superabsorbent polymer particles in the dry state, i.e., particles containing from no water up to an amount of water less than the weight of the particles. “Base polymer particles” are SAP particles prior to a surface-crosslinking process. “Surface-crosslinked SAP particles” are base polymer particles that have been subjected to a surface-crosslinking process, as described more fully hereafter. The term “particles” refers to granules, fibers, flakes, agglomerates, rods, spheres, needles, powders, films, platelets, and other shapes and forms known to persons skilled in the art of superabsorbent polymers, as well as combinations thereof. The particles can have any desired shape such as, for example, cubic, rod-like, polyhedral, spherical or semi-spherical, rounded or semi-rounded, angular, irregular, and the like. The terms “SAP gel” and “SAP hydrogel” refer to a superabsorbent polymer in the hydrated state, i.e., particles that have absorbed at least their weight in water, and typically several times their weight in water. The term “coated SAP particles” and “coated surface-crosslinked polymer particles” refer to particles of the present invention, i.e., SAP particles having a polyamine coating comprising a polyamine and a surface crosslinking agent.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers and/or other materials are collected on a forming surface, such as provided by a foraminous belt. The forming surface may include a gas-pervious material, such as spunbonded fabric material, that has been placed onto the forming surface.

The terms “elastic,” “elastomeric,” “elastically” and “elastically extensible” are used interchangeably to refer to a material or composite that generally exhibits properties which approximate the properties of natural rubber. The elastomeric material is generally capable of being extended or otherwise deformed, and then recovering a significant portion of its shape after the extension or deforming force is removed.

The term “extensible” refers to a material that is generally capable of being extended or otherwise deformed, but which does not recover a significant portion of its shape after the extension or deforming force is removed.

The terms “fluid impermeable,” “liquid impermeable,” “fluid impervious” and “liquid impervious” mean that fluid such as water or bodily fluids will not pass substantially through the layer or laminate under ordinary use conditions in a direction generally perpendicular to the plane of the layer or laminate at the point of fluid contact.

The term “health/medical absorbent articles” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “household/industrial absorbent articles” includes construction and packaging supplies, products for cleaning and disinfecting, wipes, covers, filters, towels, disposable cutting sheets, bath tissue, facial tissue, nonwoven roll goods, home-comfort products including pillows, pads, mats, cushions, masks and body care products such as products used to cleanse or treat the skin, laboratory coats, cover-alls, trash bags, stain removers, topical compositions, pet care absorbent liners, laundry soil/ink absorbers, detergent agglomerators, lipophilic fluid separators, and the like.

The terms “hydrophilic” and “wettable” are used interchangeably to refer to a material having a contact angle of water in air of less than 90 degrees. The term “hydrophobic” refers to a material having a contact angle of water in air of at least 90 degrees. For the purposes of this application, contact angle measurements are determined as set forth in Robert J. Good and Robert J. Stromberg, Ed., in “Surface and Colloid Science—Experimental Methods,” Vol. II, (Plenum Press, 1979), which is hereby incorporated by reference in a manner that is consistent herewith.

The term “increased amount of extractable materials” refers to SAP particles having an amount of extractable materials that is greater than the amount present in conventional SAP particles, as determined by the method set forth below, e.g., greater than 3%, by weight. Typically, the increased amount of extractable materials is greater than 3%, and up to about 15%, by weight, of the SAP particles.

The term “layer” when used in the singular can have the dual meaning of a single element or a plurality of elements.

The term “MD” or “machine direction” refers to the orientation of the absorbent web that is parallel to the running direction of the forming fabric and generally within the plane formed by the forming surface. The term “CD” or “cross-machine direction” refers to the direction perpendicular to the MD and generally within the plane formed by the forming surface. Both MD and CD generally define a plane that is parallel to the forming surface. The term “ZD ” or “Z-direction” refers to the orientation that is perpendicular to the plane formed by the MD and CD.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. In the particular case of a coform process, the meltblown fiber stream intersects with one or more material streams that are introduced from a different direction. Thereafter, the meltblown fibers and other materials are carried by the high velocity gas stream and are deposited on a collecting surface. The distribution and orientation of the meltblown fibers within the formed web is dependent on the geometry and process conditions. Under certain process and equipment conditions, the resulting fibers can be substantially “continuous,” defined as having few separations, broken fibers or tapered ends when multiple fields of view are examined through a microscope at 10× or 20× magnification. When “continuous” melt blown fibers are produced, the sides of individual fibers will generally be parallel with minimal variation in fiber diameter within an individual fiber length. In contrast, under other conditions, the fibers can be overdrawn and strands can be broken and form a series of irregular, discrete fiber lengths and numerous broken ends. Retraction of the once attenuated broken fiber will often result in large clumps of polymer.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polyamine coating” refers to a coating on the surface of an SAP particle, wherein the coating comprises (a) a polymer containing at least two, and typically a plurality, of primary, and/or secondary, and/or tertiary, and/or quaternary nitrogen atoms, (b) water, (c) an optional cosolvent, and (d) an optional crosslinking agent. At least a portion of the water and optional cosolvent typically evaporate from the coating during the step of applying the coating to the SAP particles. The cosolvent is capable of transforming the polyamine-coated SAP surface from hydrophilic to hydrophobic.

The term “polyolefin” as used herein generally includes, but is not limited to, materials such as polyethylene, polypropylene, polyisobutylene, polystyrene, ethylene vinyl acetate copolymer and the like, the homopolymers, copolymers, terpolymers, etc., thereof, and blends and modifications thereof. The term “polyolefin” shall include all possible structures thereof, which includes, but is not limited to, isotatic, synodiotactic and random symmetries. Copolymers include random and block copolymers.

The term “sports accessory absorbent articles” includes headbands, wrist bands and other aids for absorption of perspiration, absorptive windings for grips and handles of sports equipment, and towels or absorbent wipes for cleaning and drying off equipment during use.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stretchable” refers to materials which may be extensible or which may be elastically extensible.

The terms “superabsorbent” and “superabsorbent polymer” refer to water-swellable, water-insoluble organic or inorganic materials capable, under the most favorable conditions, of absorbing at least about 10 times their weight, or at least about 15 times their weight, or at least about 25 times their weight in an aqueous solution containing 0.9 weight percent sodium chloride. In contrast, “absorbent materials” are capable, under the most favorable conditions, of absorbing at least 5 times their weight of an aqueous solution containing 0.9 weight percent sodium chloride.

The terms “surface treated” and “surface crosslinked” refer to an SAP, i.e., base polymer, particle having its molecular chains present in the vicinity of the particle surface crosslinked by a compound applied to the surface of the particle. The term “surface crosslinking” means that the level of functional crosslinks in the vicinity of the surface of the base polymer particle generally is higher than the level of functional crosslinks in the interior of the base polymer particle. As used herein, “surface” describes the outer-facing boundaries of the particle. For porous SAP particles, exposed internal surface also are included in the definition of surface.

The term “target zone” refers to an area of an absorbent core where it is particularly desirable for the majority of a fluid insult, such as urine, menses, or bowel movement, to initially contact. In particular, for an absorbent core with one or more fluid insult points in use, the insult target zone refers to the area of the absorbent core extending a distance equal to 15% of the total length of the composite from each insult point in both directions.

The term “thermoplastic” describes a material that softens when exposed to heat and which substantially returns to a non-softened condition when cooled to room temperature.

These terms may be defined with additional language in the remaining portions of the specification.

### DETAILED DESCRIPTION

An absorbent article of the present invention can have a topsheet, a backsheet, and an absorbent core disposed between the topsheet and the backsheet. The absorbent core includes superabsorbent polymer (SAP) particles comprising a base polymer having an amount of extractable material greater than 3%, by weight, and having a surface coating comprising a polyamine. In some aspects, at least one of the topsheet, backsheet, and absorbent core is stretchable. In other aspects, the absorbent core can comprise layers, at least one of which includes substantially the superabsorbent polymer particles and at least one of which includes substantially fluff.

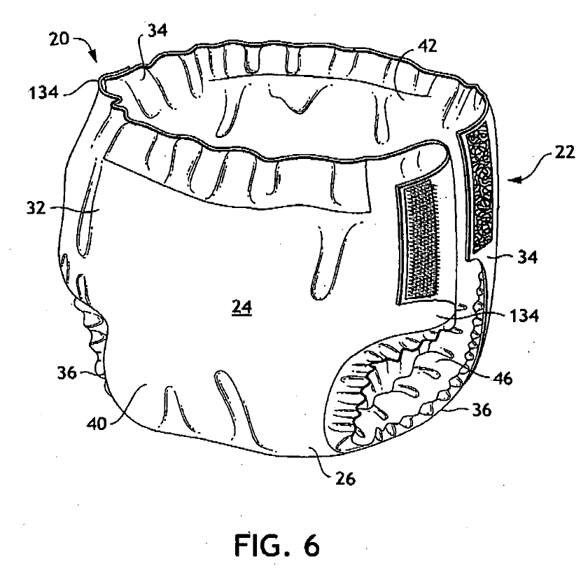

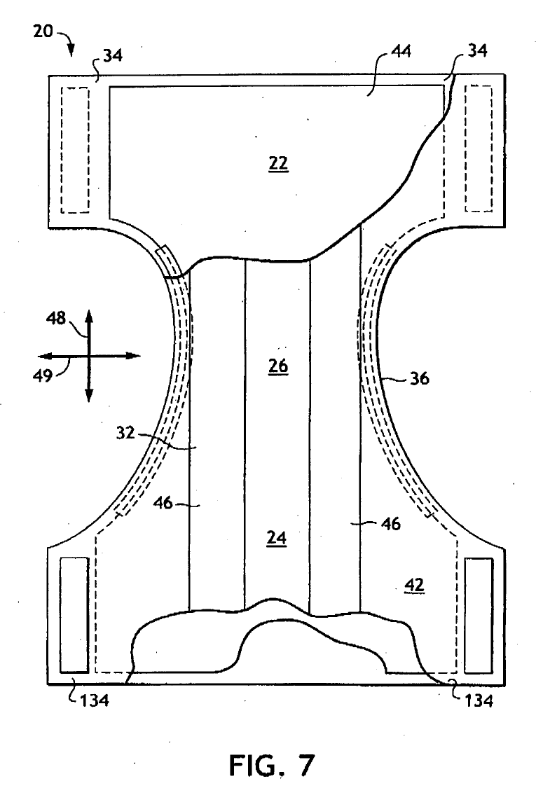

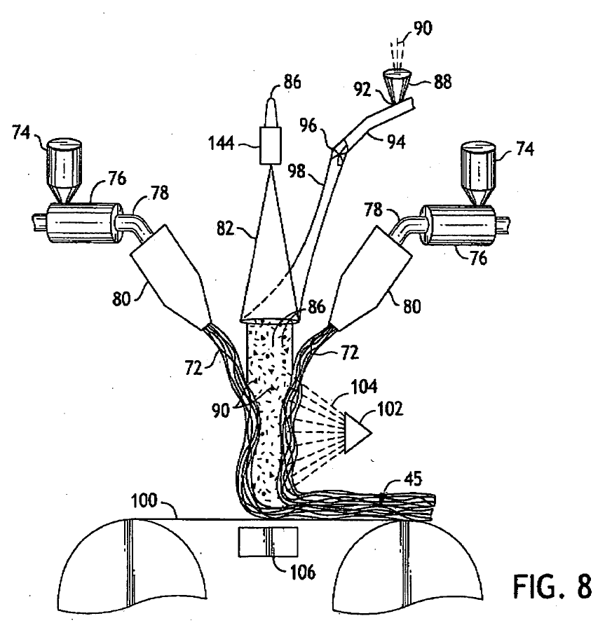

To gain a better understanding of the present invention, attention is directed to FIG. 6 and FIG. 7 for exemplary purposes showing a training pant of the present invention. It is understood that the present invention is suitable for use with various other absorbent articles, including but not limited to other personal care absorbent articles, health/medical absorbent articles, household/industrial absorbent articles, and the like, without departing from the scope of the present invention.

Various materials and methods for constructing training pants are disclosed in PCT Patent Application WO 00/37009 published Jun. 29, 2000 by A. Fletcher et al.; U.S. Pat. No. 4,940,464 to Van Gompel et al.; U.S. Pat. No. 5,766,389 to Brandon et al., and U.S. Pat. No. 6,645,190 to Olson et al., all of which are incorporated herein by reference in a manner that is consistent herewith.

FIG. 6 illustrates a training pant in a partially fastened condition, and FIG. 7 illustrates a training pant in an opened and unfolded state. The training pant defines a longitudinal direction 48 that extends from the front of the training pant when worn to the back of the training pant. Perpendicular to the longitudinal direction 48 is a lateral direction 49.

The pair of training pants defines a front region 22, a back region 24, and a crotch region 26 extending longitudinally between and interconnecting the front and back regions. The pant also defines an inner surface adapted in use (e.g., positioned relative to the other components of the pant) to be disposed toward the wearer, and an outer surface opposite the inner surface. The training pant has a pair of laterally opposite side edges and a pair of longitudinally opposite waist edges.

The illustrated pant 20 may include a chassis 32, a pair of laterally opposite front side panels 34 extending laterally outward at the front region 22 and a pair of laterally opposite back side panels 134 extending laterally outward at the back region 24.

The chassis 32 includes a backsheet 40 and a topsheet 42 that may be joined to the backsheet 40 in a superimposed relation therewith by adhesives, ultrasonic bonds, thermal bonds or other conventional techniques. The chassis 32 may further include an absorbent core 44 such as shown in FIG. 7 disposed between the backsheet 40 and the topsheet 42 for absorbing fluid body exudates exuded by the wearer, and may further include a pair of containment flaps 46 secured to the topsheet 42 or the absorbent core 44 for inhibiting the lateral flow of body exudates.

The backsheet 40, the topsheet 42 and the absorbent core 44 may be made from many different materials known to those skilled in the art. All three layers, for instance, may be extensible and/or elastically extensible. Further, the stretch properties of each layer may vary in order to control the overall stretch properties of the product.

The backsheet 40, for instance, may be breathable and/or may be fluid impermeable. The backsheet 40 may be constructed of a single layer, multiple layers, laminates, spunbond fabrics, films, meltblown fabrics, elastic netting, microporous webs or bonded-carded-webs. The backsheet 40, for instance, can be a single layer of a fluid impermeable material, or alternatively can be a multi-layered laminate structure in which at least one of the layers is fluid impermeable.

The backsheet 40 can be biaxially extensible and optionally biaxially elastic. Elastic non-woven laminate webs that can be used as the backsheet 40 include a non-woven material joined to one or more gatherable non-woven webs or films. Stretch Bonded Laminates (SBL) and Neck Bonded Laminates (NBL) are examples of elastomeric composites.

Examples of suitable nonwoven materials are spunbond-meltblown fabrics, spunbond-meltblown-spunbond fabrics, spunbond fabrics, or laminates of such fabrics with films, or other nonwoven webs. Elastomeric materials may include cast or blown films, meltblown fabrics or spunbond fabrics composed of polyethylene, polypropylene, or polyolefin elastomers, as well as combinations thereof. The elastomeric materials may include PEBAX elastomer (available from AtoFina Chemicals, Inc., a business having offices located in Philadelphia, Pa. U.S.A.), HYTREL elastomeric polyester (available from Invista, a business having offices located in Wichita, Kans. U.S.A.), KRATON elastomer (available from Kraton Polymers, a business having offices located in Houston, Tex., U.S.A.), or strands of LYCRA elastomer (available from Invista), or the like, as well as combinations thereof. The backsheet 40 may include materials that have elastomeric properties through a mechanical process, printing process, heating process or chemical treatment. For example, such materials may be apertured, creped, neck-stretched, heat activated, embossed, and micro-strained, and may be in the form of films, webs, and laminates.

One example of a suitable material for a biaxially stretchable backsheet 40 is a breathable elastic film/nonwoven laminate, such as described in U.S. Pat. No. 5,883,028, to Morman et al., incorporated herein by reference in a manner that is consistent herewith. Examples of materials having two-way stretchability and retractability are disclosed in U.S. Pat. No. 5,116,662 to Morman and U.S. Pat. No. 5,114,781 to Morman, each of which is incorporated herein by reference in a manner that is consistent herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The topsheet 42 is suitably compliant, soft-feeling and non-irritating to the wearer's skin. The topsheet 42 is also sufficiently liquid permeable to permit liquid body exudates to readily penetrate through its thickness to the absorbent core 44. A suitable topsheet 42 may be manufactured from a wide selection of web materials, such as porous foams, reticulated foams, apertured plastic films, woven and non-woven webs, or a combination of any such materials. For example, the topsheet 42 may include a meltblown web, a spunbonded web, or a bonded-carded-web composed of natural fibers, synthetic fibers or combinations thereof. The topsheet 42 may be composed of a substantially hydrophobic material, and the hydrophobic material may optionally be treated with a surfactant or otherwise processed to impart a desired level of wettability and hydrophilicity.

The topsheet 42 may also be extensible and/or elastomerically extensible. Suitable elastomeric materials for construction of the topsheet 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio U.S.A.), or PEBAX elastomers. The topsheet 42 can also be made from extensible materials such as those described in U.S. Pat. No. 6,552,245 to Roessler et al. which is incorporated herein by reference in a manner that is consistent herewith. The topsheet 42 can also be made from biaxially stretchable materials as described in U.S. Pat. No. 6,641,134 filed to Vukos et al. which is incorporated herein by reference in a manner that is consistent herewith.

The article 20 can optionally further include a surge management layer which may be located adjacent the absorbent core 44 and attached to various components in the article 20 such as the absorbent core 44 or the topsheet 42 by methods known in the art, such as by using an adhesive. In general, a surge management layer helps to quickly acquire and diffuse surges or gushes of liquid that may be rapidly introduced into the absorbent structure of the article. The surge management layer can temporarily store the liquid prior to releasing it into the storage or retention portions of the absorbent core 44. Examples of suitable surge management layers are described in U.S. Pat. No. 5,486,166 to Bishop et al.; U.S. Pat. No. 5,490,846 to Ellis et al.; and U.S. Pat. No. 5,820,973 to Dodge et al., each of which is incorporated herein by reference in a manner that is consistent herewith.

The article 20 can further comprise an absorbent core 44. The absorbent core 44 may have any of a number of shapes. For example, it may have a 2-dimensional or 3-dimensional configuration, and may be rectangular shaped, triangular shaped, oval shaped, race-track shaped, I-shaped, generally hourglass shaped, T-shaped and the like. It is often suitable for the absorbent core 44 to be narrower in the crotch portion 26 than in the rear 24 or front 22 portion(s). The absorbent core 44 can be attached in an absorbent article, such as to the backsheet 40 and/or the topsheet 42 for example, by bonding means known in the art, such as ultrasonic, pressure, adhesive, aperturing, heat, sewing thread or strand, autogenous or self-adhering, hook-and-loop, or any combination thereof.

In some aspects, the absorbent core 44 can have a significant amount of stretchability. For example, the absorbent core 44 can comprise a matrix of fibers which includes an operative amount of elastomeric polymer fibers. Other methods known in the art can include attaching superabsorbent polymer particles to a stretchable film, utilizing a nonwoven substrate having cuts or slits in its structure, and the like.

The absorbent core 44 can be formed using methods known in the art. While not being limited to the specific method of manufacture, the absorbent core can utilize a meltblown process and can further be formed on a coform line. Exemplary meltblown processes are described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. No. 3,849,241 to Butin et al. and U.S. Pat. No. 5,350,624 to Georger et al., all of which are incorporated herein by reference in a manner that is consistent herewith.

To form “coform” materials, additional components are mixed with the meltblown fibers as the fibers are deposited onto a forming surface. For example, the superabsorbent polymer particles of the present invention and fluff, such as wood pulp fibers, may be injected into the meltblown fiber stream so as to be entrapped and/or bonded to the meltblown fibers. Exemplary coform processes are described in U.S. Pat. No. 4,100,324 to Anderson et al.; U.S. Pat. No. 4,587,154 to Hotchkiss et al.; U.S. Pat. No. 4,604,313 to McFarland et al.; U.S. Pat. No. 4,655,757 to McFarland et al.; U.S. Pat. No. 4,724,114 to McFarland et al.; U.S. Pat. No. 4,100,324 to Anderson et al.; and U.K. Patent GB 2,151,272 to Minto et al., each of which is incorporated herein by reference in a manner that is consistent herewith. Absorbent, elastomeric meltblown webs containing high amounts of superabsorbent are described in U.S. Pat. No. 6,362,389 to D. J. McDowall, and absorbent, elastomeric meltblown webs containing high amounts of superabsorbent and low superabsorbent shakeout values are described in pending U.S. patent application Ser. No. 10/883174 to X. Zhang et al., each of which is incorporated herein by reference in a manner that is consistent herewith.

One example of a method of forming an absorbent core 44 for use in the present invention is illustrated in FIG. 8. The dimensions of the apparatus in FIG. 8 are described herein by way of example. Other types of apparatus having different dimensions and/or different structures may also be used to form the absorbent core 44. As shown in FIG. 8, elastomeric material 72 in the form of pellets can be fed through two pellet hoppers 74 into two single screw extruders 76 that each feed a spin pump 78. The elastomeric material 72 may be a multicomponent elastomer blend available under the trade designation VISTMAXX 2370 from ExxonMobil Chemical Company (a business having offices located in Houston, Tex. U.S.A.), as well as others mentioned herein. Each spin pump 78 feeds the elastomeric material 72 to a separate meltblown die 80. Each meltblown die 80 may have 30 holes per inch (hpi). The die angle may be adjusted anywhere between 0 and 70 degrees from horizontal, and is suitably set at about 45 degrees. The forming height may be at a maximum of about 16 inches, but this restriction may differ with different equipment.

A chute 82 having a width of about 24 inches wide may be positioned between the meltblown dies 80. The depth, or thickness, of the chute 82 may be adjustable in a range from about 0.5 to about 1.25 inches, or from about 0.75 to about 1.0 inch. A picker 144 connects to the top of the chute 82. The picker 144 is used to fiberize the pulp fibers 86. The picker 144 may be limited to processing low strength or debonded (treated) pulps, in which case the picker 144 may limit the illustrated method to a very small range of pulp types. In contrast to conventional hammermills that use hammers to impact the pulp fibers repeatedly, the picker 144 uses small teeth to tear the pulp fibers 86 apart. Suitable pulp fibers 86 for use in the method illustrated in FIG. 8 include those mentioned herein, such as NB480 (available from Weyerhaeuser Co., a business having offices located in Federal Way, Wash. U.S.A.).

At an end of the chute 82 opposite the picker 144 is a superabsorbent polymer particles feeder 88. The feeder 88 pours the superabsorbent polymer particles 90 of the present invention into a hole 92 in a pipe 94 which then feeds into a blower fan 96. Past the blower fan 96 is a length of 4-inch diameter pipe 98 sufficient for developing a fully developed turbulent flow at about 5,000 feet per minute, which allows the superabsorbent polymer particles 90 to become distributed. The pipe 98 widens from a 4-inch diameter to the 24-inch by 0.75-inch chute 82, at which point the superabsorbent polymer particles 90 mixes with the pulp fibers 86 and the mixture falls straight down and gets mixed on either side at an approximately 45-degree angle with the elastomeric material 72. The mixture of superabsorbent polymer particles 90, pulp fibers 86, and elastomeric material 72 falls onto a wire conveyor 100 moving from about 14 to about 35 feet per minute. However, before hitting the wire conveyor 100, a spray boom 102 optionally sprays an aqueous surfactant mixture 104 in a mist through the mixture, thereby rendering the resulting absorbent core 44 wettable. The surfactant mixture 104 may be a 1:3 mixture of GLUCOPON 220 UP (available from Cognis Corporation having a place of business in Cincinnati, Ohio, U.S.A.) and AHCOVEL Base N-62 (available from Uniqema, having a place of business in New Castle, Del., U.S.A.). An under wire vacuum 106 is positioned beneath the conveyor 100 to assist in forming the absorbent core 44.

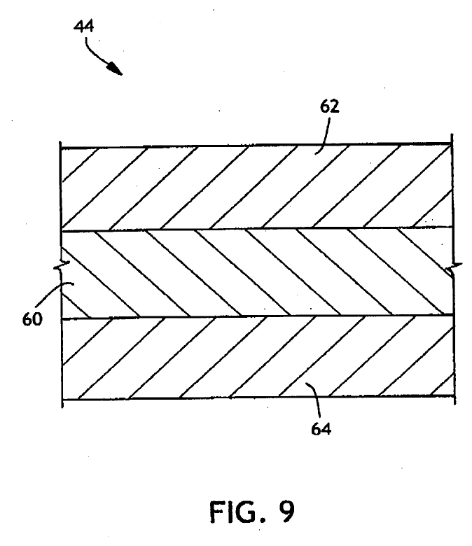

In general, the absorbent core 44 is often a unitary structure comprising a substantially uniform distribution of superabsorbent polymer particles, fibers, and any other optional additives. However, referring to FIG. 9, in some aspects, the absorbent core 44 may be further enhanced through structural modifications when combined with superabsorbent polymer particles of the present invention. For example, providing a layer 65 comprising substantially superabsorbent polymer particles of the present invention sandwiched between layers 67 and 64 comprising substantially fluff fibers, such as NB480, or other natural or synthetic fibers can result in an absorbent core 44 having improved absorbent properties, such as fluid insult intake rate, when compared to a structure comprising a substantially uniform distribution of the superabsorbent polymer particles and fluff fibers. Such layering can occur in the z-direction of the absorbent core 44 and may optionally cover the entire x-y area. However, the layers 65 through 64 need not be discreet from one another. For example, in some aspects, the z-directional middle portion 65 of the absorbent core need only contain a higher superabsorbent polymer particles percentage (e.g., at least about 10% by weight higher) than the top layer 67 and/or bottom layer 64 of the absorbent core 44. Desirably, the layers 65 through 64 are present in the area of the absorbent core 44 that is within an insult target zone.

As referenced above, the absorbent core 44 also includes absorbent material, such as superabsorbent polymer particles and/or fluff. Additionally, the superabsorbent polymer particles can be operatively contained within a matrix of fibers, such as polymeric fibers. Accordingly, the absorbent core 44 can comprise a quantity of superabsorbent polymer particles and/or fluff contained within a matrix of fibers. In some aspects, the amount of superabsorbent polymer particles in the absorbent core 44 can be at least about 10% by weight of the core, such as at least about 30%, or at least about 60% by weight or at least about 90%, or between about 10% and about 99% by weight of the core, or between about 30% to about 90% by weight of the core to provide improved benefits. Optionally, the amount of superabsorbent polymer particles can be at least about 95% by weight of the core. In other aspects, the absorbent core 44 can comprise about 35% or less by weight fluff, such as about 20% or less, or 10% or less by weight fluff.

It should be understood that the present invention is not restricted to use with superabsorbent polymer particles and/or fluff. In some aspects, the absorbent core 44 may additionally or alternatively include materials such as surfactants, ion exchange resin particles, moisturizers, emollients, perfumes, natural fibers, synthetic fibers, fluid modifiers, odor control additives, and combinations thereof. Alternatively, the absorbent core 44 can include a foam.

In order to function well, the absorbent core 44 can have certain desired properties to provide improved performance as well as greater comfort and confidence among the user. For instance, the absorbent core 44 can have corresponding configurations of absorbent capacities, densities, basis weights and/or sizes which are selectively constructed and arranged to provide desired combinations of absorbency properties such as liquid intake rate, absorbent capacity, liquid distribution or fit properties such as shape maintenance and aesthetics. Likewise, the components can have desired wet to dry strength ratios, mean flow pore sizes, permeabilities and elongation values.

As mentioned above, the absorbent core 44 can optionally include elastomeric polymer fibers. The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc. under the trade designation KRATON elastomeric resin or from Dexco, a division of ExxonMobil Chemical Company under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from Invista Corporation under the trade name LYCRA polyurethane, and ESTANE available from Noveon, Inc., a business having offices located in Cleveland, Ohio U.S.A.; thermoplastic elastic polyamides, including polyether block amides available from AtoFina Chemicals, Inc. (a business having offices located in Philadelphia, Pa. U.S.A.) under the trade name PEBAX; polyether block amide; thermoplastic elastic polyesters, including those available from E. I. Du Pont de Nemours Co., under the trade name HYTREL, and ARNITEL from DSM Engineering Plastics (a business having offices located in Evansville, Ind., U.S.A.) and single-site or metallocene-catalyzed polyolefins having a density of less than about 0.89 grams/cubic centimeter, available from Dow Chemical Co. (a business having offices located in Freeport, Tex. U.S.A.) under the trade name AFFINITY; and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS and SEPS. In these copolymers the A blocks are polystyrene and the B blocks are a rubbery component. Generally, these triblock copolymers have molecular weights that can vary from the low thousands to hundreds of thousands, and the styrene content can range from 5% to 75% based on the weight of the triblock copolymer. A diblock copolymer is similar to the triblock, but is of an AB structure. Suitable diblocks include styrene-isoprene diblocks, which have a molecular weight of approximately one-half of the triblock molecular weight having the same ratio of A blocks to B blocks.

In desired arrangements, the polymer fibers can include at least one material selected from the group consisting of styrenic block copolymers, elastic polyolefin polymers and co-polymers and EVA/EMA type polymers.

In some particular arrangements, for example, the elastomeric material of the polymer fibers can include various commercial grades of low crystallinity, lower molecular weight metallocene polyolefins, available from ExxonMobil Chemical Company (a company having offices located in Houston, Tex., U.S.A.) under the VISTAMAXX trade designation. Some VISTAMAXX materials are believed to be metallocene propylene ethylene co-polymer. For example, in one aspect the elastomeric polymer can be VISTAMAXX PLTD 2210. In other aspects, the elastomeric polymer can be VISTAMAXX PLTD 1778. In a particular aspect, the elastomeric polymer is VISTAMAXX 2370. Another optional elastomeric polymer is KRATON blend G 2755 from Kraton Inc. The KRATON material is believed to be a blend of styrene ethylene-butylene styrene polymer, ethylene waxes and tackifying resins.

In some aspects, the elastomeric polymer fibers can be produced from a polymer material having a selected melt flow rate (MFR). In a particular aspect, the MFR can be up to a maximum of about 300. Alternatively, the MFR can be up to about 230 or 250. In another aspect, the MFR can be a minimum of not less than about 9, or not less than 20. The MFR can alternatively be not less than about 50 to provide desired performance. The described melt flow rate has the units of grams flow per 10 minutes (g/10 min). The parameter of melt flow rate is well known, and can be determined by conventional techniques, such as by employing test ASTM D 1238 70 “extrusion plastometer” Standard Condition “L” at 230° C. and 2.16 kg applied force.

As referenced above, the polymer fibers of the absorbent core 44 can include an amount of a surfactant. The surfactant can be combined with the polymer fibers of the absorbent core in any operative manner. Various techniques for combining the surfactant are conventional and well known to persons skilled in the art. For example, the surfactant may be compounded with the polymer employed to form a meltblown fiber structure. In a particular feature, the surfactant may be configured to operatively migrate or segregate to the outer surface of the fibers upon the cooling of the fibers. Alternatively, the surfactant may be applied to or otherwise combined with the polymer fibers after the fibers have been formed.

The polymer fibers can include an operative amount of surfactant, based on the total weight of the fibers and surfactant. In some aspects, the polymer fibers can include at least a minimum of about 0.1% by weight surfactant, as determined by water extraction. The amount of surfactant can alternatively be at least about 0.15% by weight, and can optionally be at least about 0.2% by weight to provide desired benefits. In other aspects, the amount of surfactant can be generally not more than a maximum of about 2% by weight, such as not more than about 1% by weight, or not more than about 0.5% by weight to provide improved performance.

If the amount of surfactant is outside the desired ranges, various disadvantages can occur. For example, an excessively low amount of surfactant may not allow fibers, such as hydrophobic meltblown fibers, to wet with the absorbed fluid. In contrast, an excessively high amount of surfactant may allow the surfactant to wash off from the fibers and undesirably interfere with the ability of the absorbent core to transport fluid, or may adversely affect the attachment strength of the absorbent core to the absorbent article. Where the surfactant is compounded or otherwise internally added to the polymer fibers, an excessively high level of surfactant can create conditions that cause poor formation of the polymer fibers and interfiber bonds.

In some configurations, the surfactant can include at least one material selected from the group that includes polyethylene glycol ester condensates and alkyl glycoside surfactants. For example, the surfactant can be a GLUCOPON surfactant, available from Cognis Corporation, which can be composed of 40% water, and 60% d-glucose, decyl, octyl ethers and oligomerics.

In other aspects of the invention, the surfactant can be in the form of a sprayed-on surfactant comprising a water/surfactant solution which includes 16 liters of hot water (about 45° C. to 50° C.) mixed with 0.20 kg of GLUCOPON 220 UP surfactant available from Cognis Corporation and 0.36 kg of AHCHOVEL Base N-62 surfactant available from Uniqema. When employing a sprayed-on surfactant, a relatively lower amount of sprayed-on surfactant may be desirable to provide the desired containment of the superabsorbent polymer particles. Excessive amounts of the fluid surfactant may hinder the desired attachment of the superabsorbent polymer particles to the molten, elastomeric meltblown fibers, for example.

An example of an internal surfactant or wetting agent that can be compounded with the elastomeric fiber polymer can include a MAPEG DO 400 PEG (polyethylene glycol) ester, available from BASF (a business having offices located in Freeport, Tex., U.S.A.). Other internal surfactants can include a polyether, a fatty acid ester, a soap or the like, as well as combinations thereof.

As referenced above, the absorbent core 44 can optionally include fluff, such as cellulosic fibers. Such cellulosic fibers may include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, other typical wood pulps, cellulose acetate, debonded chemical wood pulp, and combinations thereof. Pulps derived from both deciduous and coniferous trees can be used. Additionally, the cellulosic fibers may include such hydrophilic materials as natural plant fibers, milkweed floss, cotton fibers, microcrystalline cellulose, microfibrillated cellulose, or any of these materials in combination with wood pulp fibers. Suitable cellulosic fluff fibers can include, for example, NB480 (available from Weyerhaeuser Co.); NB416, a bleached southern softwood Kraft pulp (available from Weyerhaeuser Co.); CR 54, a bleached southern softwood Kraft pulp (available from Bowater Inc., a business having offices located in Greenville, S.C. U.S.A.).; SULPHATATE HJ, a chemically modified hardwood pulp (available from Rayonier Inc., a business having offices located in Jesup, Ga. U.S.A.); NF 405, a chemically treated bleached southern softwood Kraft pulp (available from Weyerhaeuser Co.); and CR 1654, a mixed bleached southern softwood and hardwood Kraft pulp (available from Bowater Inc.)

As referenced above, the absorbent core 44 also includes a desired amount of superabsorbent polymer particles (SAPs) of the present invention. SAP particles typically are polymers of unsaturated carboxylic acids or derivatives thereof. These polymers are rendered water insoluble, but water swellable, by crosslinking the polymer with a di- or polyfunctional internal crosslinking agent. These internally crosslinked polymers are at least partially neutralized and contain pendant anionic carboxyl groups on the polymer backbone that enable the polymer to absorb aqueous fluids, such as body fluids. Typically, the SAP particles are subjected to a post-treatment to crosslink the pendant anionic carboxyl groups on the surface of the particle.

SAPs are manufactured by known polymerization techniques, desirably by polymerization in aqueous solution by gel polymerization. The products of this polymerization process are aqueous polymer gels, i.e., SAP hydrogels, that are reduced in size to small particles by mechanical forces, then dried using drying procedures and apparatus known in the art. The drying process is followed by pulverization of the resulting SAP particles to the desired particle size.

To improve the fluid absorption profile, SAP particles are optimized with respect to one or more of absorption capacity, absorption rate, acquisition time, gel strength, and/or permeability. Optimization allows a reduction in the amount of cellulosic fiber in an absorbent article, which results in a thinner article. However, it is difficult to impossible to maximize all of these absorption profile properties simultaneously.

One method of optimizing the fluid absorption profile of SAP particles is to provide SAP particles of a predetermined particle size distribution. In particular, particles too small in size swell after absorbing a fluid and can block the absorption of further fluid. Particles too large in size have a reduced surface area which decreases the rate of absorption.

Therefore, the particle size distribution of the SAP particles is such that fluid permeability, absorption, and retention by the SAP particles is maximized. Any subsequent process that agglomerates the SAP particles to provide oversized particles should be avoided. In particular, agglomeration of SAP particles increases apparent particle size, which reduces the surface area of the SAP particles, and in turn adversely affects absorption of an aqueous fluid by the SAP particles.

The present invention is directed to overcoming problems encountered in improving the absorption profile of surface-crosslinked SAP particles because improving one property often is detrimental to a second property. The present SAP particles can maintain the conflicting properties of a high centrifuge retention capacity (CRC), absorbance under load (AUL), an excellent gel bed permeability (GBP), and a good gel integrity (GI). These problems are overcome in some aspects because of a polyamine coating as well as, in part, because of (a) the reduced tendency of the present SAP particles to agglomerate, and (b) the delayed swelling of the particles after an insult, i.e., contact, with an aqueous liquid.

In order to use an increased amount of SAP particles, and a decreased amount of cellulose, in absorbent products, it is important to maintain a high SAP liquid permeability. In particular, the permeability of a SAP particle hydrogel layer formed by swelling in the presence of a body fluid is very important to overcome the problem of leakage from the product. A lack of permeability directly impacts the ability of SAP particle hydrogel layers to acquire and distribute body fluids.

Polyamines are known to adhere to cellulose (i.e., fluff), and polyamine-coated SAPs have some improved permeability, as measured in the bulk, for a lower capacity SAP. Coating of SAP particles with uncrosslinked polyamines improves adhesion to cellulose fibers because of the high flexibility of polyamine molecules. Desirably, covalent bonding of the polyamine to the SAP particles is avoided because the degree of SAP particle crosslinking is increased and the absorptive capacity of the particles is reduced. Moreover, covalent bonding of polyamine to the SAP particle surface typically occurs at a temperature greater than 150° C., which adversely affects the color of the SAP particles, and, ultimately, consumer acceptance of the absorbent article.

In accordance with the present invention, surface-crosslinked SAP particles coated with a polyamine solution and a cosolvent (which may be optional in some aspects) are disclosed. In some aspects, the present invention can help overcome the problem of a rapid swelling of SAP particles at the point of fluid insult in a diaper, which can cause gel blocking and slow fluid intake speed. The ultimate result of gel blocking is underutilization of the SAP particles in an absorbent core. The SAP particles of the present invention have a transient hydrophobic property that reduces the problem of gel blocking because swelling of the SAP particles is delayed, as measured by wicking index, after contact with an aqueous liquid.

Some aspects of the present invention demonstrate the unexpected result that coating of surface-crosslinked SAP particles with three hydrophilic compounds, i.e., polyamine, cosolvent, and water, provides SAP particles having hydrophobic surface properties, as measured by wicking index. Further, the hydrophobicity of SAP particle surfaces can be adjusted by different variables, including relative ratios of the polyamine and cosolvent, temperature of the coating process, and use of an ionic or covalent crosslinking agent.

The present SAP particles comprise a base polymer. The base polymer can be a homopolymer or a copolymer. The identity of the base polymer is not limited as long as the polymer is an anionic polymer, i.e., contains pendant acid moieties, and is capable of swelling and absorbing at least ten times its weight in water, when in a neutralized form. Desirable base polymers are crosslinked polymers having acid groups that are at least partially in the form of a salt, generally an alkali metal or ammonium salt.

The base polymer has at least about 25% of the pendant acid moieties, e.g., carboxylic acid moieties, present in a neutralized form. Desirably, the base polymer has about 50% to about 100%, such as about 65% to about 80%, of the pendant acid moieties present in a neutralized form. In accordance with the present invention, the base polymer has a degree of neutralization (DN) of about 25 to about 100.

The base polymer of the SAP particles is a lightly crosslinked polymer capable of absorbing several times its own weight in water and/or saline. SAP particles can be made by any conventional process for preparing superabsorbent polymers and are well known to those skilled in the art. One process for preparing SAP particles is a solution polymerization method described in U.S. Pat. Nos. 4,076,663; 4,286,082; and 5,145,906, each incorporated herein by reference. Another process is an inverse suspension polymerization method described in U.S. Pat. Nos. 4,340,706; 4,497,930; 4,666,975; 4,507,438; and 4,683,274, each incorporated herein by reference.

SAP particles useful in the present invention are prepared from one or more monoethylenically unsaturated compound having at least one acid moiety, such as carboxyl, carboxylic acid anhydride, carboxylic acid salt, sulfuric acid, sulfuric acid salt, sulfonic acid, sulfonic acid salt, phosphoric acid, phosphoric acid salt, phosphonic acid, or phosphonic acid salt. SAP particles useful in the present invention desirably are prepared from one or more monoethylenically unsaturated, water-soluble carboxyl or carboxylic acid anhydride containing monomer, and the alkali metal and ammonium salts thereof, wherein these monomers desirably comprise 50 to 99.9 mole percent of the base polymer.

The base polymer of the SAP particles desirably is a lightly crosslinked acrylic resin, such as lightly crosslinked polyacrylic acid. The lightly crosslinked base polymer typically is prepared by polymerizing an acidic monomer containing an acyl moiety, e.g., acrylic acid, or a moiety capable of providing an acid group, i.e., acrylonitrile, in the presence of an internal crosslinking agent, i.e., a polyfunctional organic compound. The base polymer can contain other copolymerizable units, i.e., other monoethylenically unsaturated comonomers, well known in the art, as long as the base polymer is substantially, i.e., at least 10%, such as at least 25%, acidic monomer units, e.g., (meth)acrylic acid. To achieve the full advantage of the present invention, the base polymer contains at least 50%, such as at least 75%, or up to 100%, acidic monomer units. The other copolymerizable units can, for example, help improve the hydrophilicity of the polymer.

Ethylenically unsaturated carboxylic acid and carboxylic acid anhydride monomers useful in the base polymer include acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, itaconic acid, citraconic acid, mesaconic acid, glutaconic acid, aconitic acid, maleic acid, fumaric acid, tricarboxyethylene, and maleic anhydride.

Ethylenically unsaturated sulfonic and phosphonic acid monomers include aliphatic or aromatic vinyl sulfonic acids, such as vinylsulfonic acid, allylsulfonic acid, vinyl toluene sulfonic acid, styrene sulfonic acid, acrylic and methacrylic sulfonic acids, such as sulfoethyl acrylate, sulfoethyl methacrylate, sulfopropyl acrylate, sulfopropyl methacrylate, 2-hydroxy-3-methacryloxypropyl sulfonic acid, 2-acrylamido-2-methylpropane sulfonic acid, vinylphosphonic acid, allylphosphonic acid, and mixtures thereof.

Desirable, but nonlimiting, monomers include acrylic acid, methacrylic acid, maleic acid, fumaric acid, maleic anhydride, and the sodium, potassium, and ammonium salts thereof. An especially desirable monomer is acrylic acid.

The base polymer can contain additional monoethylenically unsaturated monomers that do not bear a pendant acid group, but are copolymerizable with monomers bearing acid groups. Such compounds include, for example, the amides and nitriles of monoethylenically unsaturated carboxylic acids, for example, acrylamide, methacrylamide, acrylonitrile, and methacrylonitrile. Examples of other suitable comonomers include, but are not limited to, vinyl esters of saturated C₁₋₄ carboxylic acids, such as vinyl formate, vinyl acetate, and vinyl propionate; alkyl vinyl ethers having at least two carbon atoms in the alkyl group, for example, ethyl vinyl ether and butyl vinyl ether; esters of monoethylenically unsaturated C₃₋₁₈ alcohols and acrylic acid, methacrylic acid, or maleic acid; monoesters of maleic acid, for example, methyl hydrogen maleate; acrylic and methacrylic esters of alkoxylated monohydric saturated alcohols, for example, alcohols having 10 to 25 carbon atoms reacted with 2 to 200 moles of ethylene oxide and/or propylene oxide per mole of alcohol; and monoacrylic esters and monomethacrylic esters of polyethylene glycol or polypropylene glycol, the molar masses (Mₙ) of the polyalkylene glycols being up to about 2,000, for example. Further suitable comonomers include, but are not limited to, styrene and alkyl-substituted styrenes, such as ethylstyrene and tert-butylstyrene, and 2-hydroxyethyl acrylate.

Polymerization of the acidic monomers, and any copolymerizable monomers, most commonly is performed by free radical processes in the presence of a polyfunctional organic compound. The base polymers are internally crosslinked to a sufficient extent such that the base polymer is water insoluble. Internal crosslinking renders the base polymer substantially water insoluble, and, in part, serves to determine the absorption capacity of the base polymer. For use in absorption applications, a base polymer is lightly crosslinked, i.e., has a crosslinking density of less than about 20%, such as less than about 10%, or about 0.01% to about 7%.

A crosslinking agent most desirably is used in an amount of less than about 7 wt %, and typically about 0.1 wt % to about 5 wt %, based on the total weight of monomers. Examples of crosslinking polyvinyl monomers include, but are not limited to, polyacrylic (or polymethacrylic) acid esters represented by the following formula (I), and bisacrylamides represented by the following formula (II):

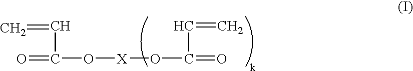

wherein X is ethylene, propylene, trimethylene, cyclohexyl, hexamethylene, 2-hydroxypropylene, —(CH₂CH₂O)ₙCH₂CH₂—, or

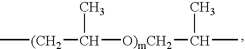

### Wherein n and m are each an integer 5 to 40, and k is 1 or 2;

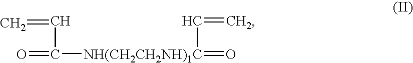

wherein 1 is 2 or 3.

The compounds of formula (I) are prepared by reacting polyols, such as ethylene glycol, propylene glycol, trimethylolpropane, 1,6-hexanediol, glycerin, pentaerythritol, polyethylene glycol, or polypropylene glycol, with acrylic acid or methacrylic acid. The compounds of formula (II) are obtained by reacting polyalkylene polyamines, such as diethylenetriamine and triethylenetetramine, with acrylic acid.

Specific internal crosslinking agents include, but are not limited to, 1,4-butanediol diacrylate, 1,4-butanediol dimethacrylate, 1,3-butylene glycol diacrylate, 1,3-butylene glycol dimethacrylate, diethylene glycol diacrylate, diethylene glycol dimethacrylate, ethoxylated bisphenol A diacrylate, ethoxylated bisphenol A dimethacrylate, ethylene glycol dimethacrylate, 1,6-hexanediol diacrylate, 1,6-hexanediol dimethacrylate, neopentyl glycol dimethacrylate, polyethylene glycol diacrylate, polyethylene glycol dimethacrylate, triethylene glycol diacrylate, triethylene glycol dimethacrylate, tripropylene glycol diacrylate, tetraethylene glycol diacrylate, tetraethylene glycol dimethacrylate, dipentaerythritol pentaacrylate, penta-erythritol tetraacrylate, pentaerythritol triacrylate, trimethylolpropane triacrylate, trimethylolpropane trimethacrylate, tris(2-hydroxyethyl)-isocyanurate triacrylate, ethoxylated trimethylolpropane triacrylate (ETMPTA), e.g., ETMPTA ethyoxylated with 15 moles of ethylene oxide (EO) on average, tris(2-hydroxyethyl)isocyanurate trimethyacrylate, divinyl esters of a polycarboxylic acid, diallyl esters of a polycarboxylic acid, triallyl terephthalate, diallyl maleate, diallyl fumarate, hexamethylenebismaleimide, trivinyl trimellitate, divinyl adipate, diallyl succinate, a divinyl ether of ethylene glycol, cyclopentadiene diacrylate, a tetraallyl ammonium halide, divinyl benzene, divinyl ether, diallyl phthalate, or mixtures thereof. Especially desirable internal crosslinking agents are N,N′-methylenebisacrylamide, N,N′-methylenebismethacrylamide, ethylene glycol dimethacrylate, and trimethylolpropane triacrylate.

The base polymer can be any internally crosslinked polymer having pendant acid moieties that acts as a SAP in its neutralized form. Examples of base polymers include, but are not limited to, polyacrylic acid, hydrolyzed starch-acrylonitrile graft copolymers, starch-acrylic acid graft copolymers, saponified vinyl acetate-acrylic ester copolymers, hydrolyzed acrylonitrile copolymers, hydrolyzed acrylamide copolymers, ethylene-maleic anhydride copolymers, isobutylene-maleic anhydride copolymers, poly(vinylsulfonic acid), poly(vinylphosphonic acid), poly(vinylphosphoric acid), poly(vinylsulfuric acid), sulfonated polystyrene, poly-(aspartic acid), poly(lactic acid), and mixtures thereof. The desirable base polymer is a homopolymer or copolymer of acrylic acid or methacrylic acid.

The free radical polymerization is initiated by an initiator or by electron beams acting on a polymerizable aqueous mixture. Polymerization also can be initiated in the absence of such initiators by the action of high energy radiation in the presence of photoinitiators.

Useful polymerization initiators include, but are not limited to, compounds that decompose into free radicals under polymerization conditions, for example, peroxides, hydroperoxides, persulfates, azo compounds, and redox catalysts. Water-soluble initiators are desirable. In some cases, mixtures of different polymerization initiators are used, for example, mixtures of hydrogen peroxide and sodium peroxodisulfate or potassium peroxodisulfate. Mixtures of hydrogen peroxide and sodium peroxodisulfate can be in any proportion.

Examples of suitable organic peroxides include, but are not limited to, acetylacetone peroxide, methyl ethyl ketone peroxide, tert-butyl hydroperoxide, cumeme hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, di(2-ethylhexyl)peroxydicarbonate, dicyclohexyl peroxydicarbonate, di(4-tert-butylcyclohexyl)peroxydicarbonate, dimyristyl peroxydicarbonate, diacetyl peroxydicarbonate, an allyl perester, cumyl peroxyneodecanoate, tert-butyl per-3,5,5-trimethylhexanoate, acetylcyclohexylsulfonyl peroxide, dilauryl peroxide, dibenzoyl peroxide, and tert-amyl perneodecanoate. Particularly suitable polymerization initiators are water-soluble azo initiators, e.g., 2,2′-azobis(2-amidinopropane)dihydrochloride, 2,2′-azobis(N,N′-dimethylene)isobutyramidine dihydrochloride, 2-(carbamoylazo-isobutyronitrile, 2,2′-azobis[2-(2′-imidazolin-2-yl)propane]dihydrochloride, and 4,4′-azobis(4-cyanovaleric acid). The polymerization initiators are used, for example, in amounts of 0.01% to 5%, such as 0.05% to 2.0%, by weight, based on the monomers to be polymerized.

Polymerization initiators also include redox catalysts. In redox catalysts, the oxidizing compound comprises at least one of the above-specified per compounds, and the reducing component comprises, for example, ascorbic acid, glucose, sorbose, ammonium or alkali metal bisulfite, sulfite, thiosulfate, hyposulfite, pyrosulfite, or sulfide, or a metal salt, such as iron (II) ions or sodium hydroxymethylsulfoxylate. The reducing component of the redox catalyst desirably is ascorbic acid or sodium sulfite. Based on the amount of monomers used in the polymerization, about 3×10⁻⁶ to about 1 mol % of the reducing component of the redox catalyst system can be used, and about 0.001 to about 5.0 mol % of the oxidizing component of the redox catalyst can be used, for example.

When polymerization is initiated using high energy radiation, the initiator typically comprises a photoinitiator. Photoinitiators include, for example, α-splitters, H-abstracting systems, and azides. Examples of such initiators include, but are not limited to, benzophenone derivatives, such as Michler's ketone; phenanthrene derivatives; fluorene derivatives; anthraquinone derivatives; thioxanthone derivatives; coumarin derivatives; benzoin ethers and derivatives thereof; azo compounds, such as the above-mentioned free-radical formers, substituted hexaarylbisimidazoles, acyl-phosphine oxides; or mixtures thereof.

Examples of azides include, but are not limited to, 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulfone, N-(4-sulfonylazidophenyl)maleimide, N-acetyl-4-sulfonylazidoaniline, 4-sulfonyl-azidoaniline, 4-azidoaniline, 4-azidophenacyl bromide, p-azidobenzoic acid, 2,6-bis(p-azidobenzylidene)cyclohexanone, and 2,6-bis(p-azidobenzylidene)-4-methylcyclohexanone. Photoinitiators customarily are used, if at all, in amounts of about 0.01% to about 5%, by weight of the monomers to be polymerized.

As previously stated, the base polymer is partially neutralized. The degree of neutralization is about 25 to about 100, such as about 50 to about 90, mol %, based on monomers containing acid groups. The degree of neutralization more desirably is greater than about 60 mol %, such as about 65 to about 90 mol %, or about 65 to about 80 mol %, based on monomers containing acid groups.

Useful neutralizing agents for the base polymer include alkali metal bases, ammonia, and/or amines. Desirably, the neutralizing agent comprises aqueous sodium hydroxide, aqueous potassium hydroxide, or lithium hydroxide. However, neutralization also can be achieved using sodium carbonate, sodium bicarbonate, potassium carbonate, or potassium bicarbonate, or other carbonates or bicarbonates, as a solid or as a solution. Primary, secondary, and/or tertiary amines can be used to neutralize the base polymer.

Neutralization of the base polymer can be performed before, during, or after the polymerization in a suitable apparatus for this purpose. The neutralization is performed, for example, directly in a kneader used for polymerization of the monomers.

In accordance with the present invention, polymerization of an aqueous monomer solution, i.e., gel polymerization, is desirable. In this method, a 10% to 70%, by weight, aqueous solution of the monomers, including the internal crosslinking agent, is neutralized in the presence of a free radical initiator. The solution polymerization is performed at 0° C. to 150° C., such as at 10° C. to 100° C., and at atmospheric, superatmospheric, or reduced pressure. The polymerization also can be conducted under a protective gas atmosphere, desirably under nitrogen.

After polymerization, the resulting hydrogel of the base polymer is dried, and the dry base polymer particles are ground and classified to a predetermined size for an optimum fluid absorption profile. In accordance with the present invention, the base polymer particles then are surface crosslinked. It should be understood that the polyamine coating process step and surface crosslinking process step are different, and impart different properties to the surfaces of the base polymer particles. The base polymer particles are surface crosslinked prior to application of the polyamine coating.

In one aspect of applying a polyamine coating to the surface-crosslinked polymer particles, a surface-crosslinking agent is applied to the surfaces of the base polymer particles. Then, the resulting polymer particles are heated for a sufficient time and at a sufficient temperature to surface-crosslink the base polymer particles. Next, a coating solution containing a polyamine dissolved in water and, in some aspects, a cosolvent, and in further aspects, a crosslinking agent, is applied to the surfaces of the surface-crosslinked SAP particles. The polyamine coating is applied to surface-crosslinked SAP particles having a temperature of about 25° C. to about 100° C., such as about 50° C. to about 100° C. The polyamine coating can be applied at a temperature, for example, of 25, 30, 35, 40, 45, 50, 55, 60, 65, 70, 75, 80, 85, 90, 95, or 100° C.

The polyamine coating is added to surface-crosslinked SAP particles after the surface-crosslinking step, wherein the surface-crosslinked SAP particles are cooling, but still warm. Accordingly, the polyamine coating is applied under the latent heat of the surface-crosslinked SAP particles. If needed, an external heat source can be used to achieve a desired polyamine-coated SAP particle temperature of up to about 100° C.

After applying the polyamine coating to the surface-crosslinked SAP particles, the coated SAP particles are mixed for about 5 to about 60 minutes to form a uniform polyamine coating on the surface-crosslinked polymer particles and provide SAP particles of the present invention. The polyamine coating is hydrophilic in the absence of a cosolvent, and is hydrophobic in the presence of a cosolvent.

The components of the polyamine coating solution can be applied to the SAP particles in any order, from one, two, or three solutions. In particular, the optional cosolvent and optional crosslinking agent can be applied to the surface-crosslinked SAP particles independent of the polyamine and independent of each other. Alternatively, the polyamine, optional cosolvent, and optional crosslinking agent can be administered and applied from a single solution.

In the surface crosslinking process, a multifunctional compound capable of reacting with the functional groups of the base polymer is applied to the surface of the base polymer particles, desirably using an aqueous solution. The aqueous solution also can contain water-miscible organic solvents, like an alcohol, such as methanol, ethanol, or i-propanol; a polyol, like ethylene glycol or propylene glycol; or acetone.

A solution of a surface-crosslinking agent is applied to the base polymer particles in an amount to wet predominantly only the outer surfaces of the base polymer particles, either before or after application of the polyamine. Surface cross-linking and drying of the base polymer particles is then performed, desirably by heating at least the wetted surfaces of the base polymer particles.

Typically, the base polymer particles are surface treated with a solution of a surface-crosslinking agent containing about 0.01% to about 4%, by weight, surface-crosslinking agent, such as about 0.4% to about 2%, by weight, surface-crosslinking agent in a suitable solvent. The solution can be applied as a fine spray onto the surfaces of freely tumbling base polymer particles at a ratio of about 1:0.01 to about 1:0.5 parts by weight base polymer particles to solution of surface-crosslinking agent. The surface-crosslinking agent is present in an amount of 0.001% to about 5%, by weight of the base polymer particles, such as 0.001% to about 0.5% by weight. To achieve the full advantage of the present invention, the surface-crosslinking agent is present in an amount of about 0.001% to about 0.2%, by weight of the base polymer particles.

Surface crosslinking of the base polymer particles and drying are achieved by heating the surface-treated base polymer particles at a suitable temperature, e.g., about 70° C. to about 200° C., such as about 105° C. to about 180° C. Suitable surface-crosslinking agents are capable of reacting with acid moieties and crosslinking polymers at the surfaces of the base polymer particles.

Nonlimiting examples of suitable surface-crosslinking agents include, but are not limited to, an alkylene carbonate, such as ethylene carbonate or propylene carbonate; a polyaziridine, such as 2,2-bishydroxymethyl butanol tris[3-(1-aziridine propionate] or bis-N-aziridinomethane; a haloepoxy, such as epichlorohydrin; a polyisocyanate, such as 2,4-toluene diisocyanate; a di- or polyglycidyl compound, such as diglycidyl phosphonates, ethylene glycol diglycidyl ether, or bischlorohydrin ethers of polyalkylene glycols; alkoxysilyl compounds; polyols such as ethylene glycol, 1,2-propanediol, 1,4-butanediol, glycerol, methyltriglycol, polyethylene glycols having an average molecular weight Mw of about 200 to about 10,000, di- and polyglycerol, pentaerythritol, sorbitol, the ethoxylates of these polyols and their esters with carboxylic acids or carbonic acid, such as ethylene carbonate or propylene carbonate; carbonic acid derivatives, such as urea, thiourea, guanidine, dicyandiamide, 2-oxazolidinone and its derivatives, bisoxazoline, polyoxazolines, di- and polyisocyanates; di- and poly-N-methylol compounds, such as methylenebis(N-methylolmethacrylamide) or melamine-formaldehyde resins; compounds having two or more blocked isocyanate groups, such as trimethylhexamethylene diisocyanate blocked with 2,2,3,6-tetramethylpiperidin-4-one; 2-hydroxyethyloxazolidinone; hydroxyalkylamides as disclosed in U.S. Pat. No. 6,239,230, incorporated herein by reference in a manner that is consistent herewith; and other surface-crosslinking agents known to persons skilled in the art.

A polyamine can be applied to the polymer particles after the surface crosslinking step has been completed. A solution containing the polyamine comprises about 5% to about 50%, by weight, of a polyamine in a suitable solvent. Typically, a sufficient amount of a solvent is present to allow the polyamine to be readily and homogeneously applied to the surfaces of the base polymer particles. In some aspects, the polymer particles may be surface-crosslinked. The solvent for the polyamine solution typically comprises water.

The amount of polyamine applied to the surfaces of the surface-crosslinked polymer particles is sufficient to coat the surface-crosslinked polymer particle surfaces. Accordingly, the amount of polyamine applied to the surfaces of the surface-crosslinked polymer particles is about 0.1% to about 2%, such as about 0.2% to about 1%, of the weight of the surface-crosslinked polymer particles. To achieve the full advantage of the present invention, the polyamine is present on the surface-crosslinked polymer particle surfaces in an amount of about 0.2% to about 0.5%, by weight of the surface-crosslinked polymer particles.

A polyamine can form an ionic bond with a surface-crosslinked polymer particle and retains adhesive forces to the surface-crosslinked particle after the surface-crosslinked polymer absorbs a fluid and swells. Desirably, an excessive amount of covalent bonds are not formed between the polyamine and the surface-crosslinked polymer particle, and interactions between the polyamine and surface-crosslinked polymer particle are intermolecular, such as electrostatic, hydrogen bonding, and van der Waals interactions. Therefore, the presence of a polyamine on surface-crosslinked SAP particles does not adversely influence the absorption profile of the surface-crosslinked SAP particles.

A polyamine useful in the present invention has at least two, and desirably a plurality, of nitrogen atoms per molecule. The polyamine typically has a weight average molecular weight (M_{w}) of about 5,000 to about 1,000,000, such as about 20,000 to about 600,000. To achieve the full advantage of the present invention, the polyamine has an M_{w} of about 100,000 to about 400,000.

In general, useful polyamines have (a) primary amino groups, (b) secondary amino groups, (c) tertiary amino groups, (d) quaternary ammonium groups, or (e) mixtures thereof. Examples of polyamines include, but are not limited to, a polyvinylamine, a polyallylamine, a polyethyleneimine, a polyalkyleneamine, a polyazetidine, a polyvinylguanidine, a poly(DADMAC), i.e., a poly(diallyl dimethyl ammonium chloride), a cationic polyacrylamide, a polyamine functionalized polyacrylate, and mixtures thereof.

Homopolymers and copolymers of vinylamine also can be used, for example, copolymers of vinylformamide and comonomers, which are converted to vinylamine copolymers. The comonomers can be any monomer capable of copolymerizing with vinylformamide. Nonlimiting examples of such monomers include, but are not limited to, acrylamide, methacrylamide, methacrylonitrile, vinylacetate, vinyl-propionate, styrene, ethylene, propylene, N-vinylpyrrolidone, N-vinylcaprolactam, N-vinylimidazole, monomers containing a sulfonate or phosphonate group, vinylglycol, acrylamido(methacrylamido)alkylene trialkyl ammonium salt, diallyl dialkylammonium salt, C₁₋₄alkyl vinyl ethers such as methyl vinyl ether, ethyl vinyl ether, isopropyl vinyl ether, n-propyl vinyl ether, t-butyl vinyl ether, N-substituted alkyl(meth)acrylamides substituted by a C₁₋₄alkyl group as, for example, N-methylacrylamide, N-isopropylacrylamide, and N,N-dimethylacrylamide, C₁₋₂₀alkyl(meth)acrylic acid esters such as methyl methacrylate, ethyl methacrylate, propyl acrylate, butyl acrylate, hydroxyethyl acrylate, hydroxyethyl methacrylate, hydroxypropyl acrylate, hydroxypropyl methacrylate, hydroxybutyl acrylate, hydroxybutyl methacrylate, 2-methylbutyl acrylate, 3-methylbutyl acrylate, 3-pentyl acrylate, neopentyl acrylate, 2-methylpentyl acrylate, hexyl acrylate, cyclohexyl acrylate, 2-ethylhexyl acrylate, phenyl acrylate, heptyl acrylate, benzyl acrylate, tolyl acrylate, octyl acrylate, 2-octyl acrylate, nonyl acrylate, and octyl methacrylate.

Specific copolymers of polyvinylamine include, but are not limited to, copolymers of N-vinylformamide and vinyl acetate, vinyl propionate, a C₁₋₄alkyl vinyl ether, a (meth)acrylic acid ester, acrylonitrile, acrylamide, or vinylpyrrolidone.

A polyamine coating is hydrophilic as applied to the surface-crosslinked polymer particles. The polyamine coating can be rendered hydrophobic by including a cosolvent in the polyamine coating process. The optional cosolvent contains at least one, and often two or three, hydroxy groups. Useful cosolvents include, but are not limited to, alcohols, diols, triols, and mixtures thereof, for example, methanol, ethanol, propyl alcohol, isopropyl alcohol, ethylene glycol, propylene glycol, oligomers of ethylene glycol, oligomers of propylene glycol, glycerin, monoalkyl ethers of propylene glycol, and similar hydroxy-containing solvents. An oligomer of ethylene glycol or propylene glycol contains two to four ethylene oxide or propylene oxide monomer units.

In some aspects, the polyamine solution is applied to the surface-crosslinked polymer particles in a manner such that the polyamine and optional cosolvent are uniformly distributed on the surfaces of the surface-crosslinked polymer particles. In addition to the polyamine and cosolvent, other optional ingredients can be applied to the surface crosslinked SAP particles. Such optional ingredients include, but are not limited to, clay and silica, for example, to impart anticaking properties to the polyamine-coated SAP particles. A clay or silica also can be added to the polyamine-coated SAP particles after application and curing of the polyamine coating.

In general, the number of covalent bonds that form between the polyamine and surface-crosslinked SAP particles is low, if present at all. A polyamine alone may impart a tack to surfaces of the base polymer particles, which leads to agglomeration or aggregation of coated base polymer particles, especially if the polyamine coating is hydrophilic. To overcome this potential problem, a crosslinking agent for a polyamine coating can be used.

Crosslinking of the polyamine coating is different from surface crosslinking. The crosslinking agent for the polyamine coating forms crosslinks between the nitrogen atoms of the polyamine. The surface crosslinking agent forms crosslinks with carboxyl groups of the base polymer. In addition, the surface crosslinking agent is applied to the base polymer and reacted prior to application of the polyamine coating. However, it should be understood that the crosslinking agent for the polyamine coating in some aspects may react with the nitrogen atoms of the polyamine and a small number of carboxyl groups of the base polymer.

The crosslinking agent for the polyamine coating can be organic or inorganic in nature. An organic crosslinking agent reacts with nitrogen atoms of the polyamine to form covalent bonds with the polyamine nitrogen atoms. An inorganic crosslinking agent forms ionic crosslinks via the nitrogen atoms of the polyamine coating. The crosslinking agents can be used individually or in admixture, e.g., a mixture of inorganic crosslinking agents, a mixture of organic crosslinking agents, or a mixture of inorganic and organic crosslinking agents.

In a particular aspect, the crosslinking agent is a solution containing a salt having (a) a polyvalent metal cation, i.e., a metal cation having a valence of two, three, or four, (b) a polyvalent anion, i.e., an anion having a valence of two or greater, or (c) both a polyvalent cation and a polyvalent anion, is applied to the surfaces of the surface-crosslinked polymer particles. In this embodiment, the salt is applied to the surface-crosslinked polymer particles independently from the polyamine in order to avoid a premature crosslinking reaction. The salt can be applied to the surface-crosslinked polymer particles prior to or after the polyamine is added to the surface of the surface-crosslinked polymer particles.

The polyvalent metal cation and polyvalent anion are capable of interacting, e.g., forming ionic crosslinks, with the nitrogen atoms of the polyamine. As a result, a tackless polyamine coating is formed on the surface of the base polymer to provide coated SAP particles of the present invention.

In accordance with the present invention, a salt applied to surfaces of the base polymer particles has a sufficient water solubility such that polyvalent metal cations and/or polyvalent anions are available to interact with the nitrogen atoms of the polyamine. Accordingly, a useful salt has a water solubility of at least 0.01 g of salt per 100 ml of water, such as at least 0.02 g per 100 ml of water.

A polyvalent metal cation of the salt has a valence of +2, +3, or +4, and can be, but is not limited to, Mg²⁺, Ca²⁺, Al³⁺, Sc³⁺, Ti⁴⁺, Mn²⁺, Fe^{2+/3+}, Co²⁺, Ni²⁺, Cu^{+/2+}, Zn²⁺, Y³⁺, Zr⁴⁺, La³⁺, Ce⁴⁺, Hf⁴⁺, Au³⁺, and mixtures thereof. Desirable cations are Mg²⁺, Ca²⁺, Al³⁺, Ti⁴⁺, Zr⁴⁺, La³⁺, and mixtures thereof, and particularly desirable cations are Al³⁺, Ti⁴⁺, Zr⁴⁺, and mixtures thereof. The anion of a salt having a polyvalent cation is not limited, as long as the salt has sufficient solubility in water. Examples of anions include, but are not limited to, chloride, bromide, and nitrate.

A polyvalent anion of the salt has a valence of −2, −3, or −4. The polyvalent anion can be inorganic or organic in chemical structure. The identity of the polyvalent anion is not limited as long as the anion is capable of interacting with the nitrogen atoms of the polyamine.

Examples of polyvalent inorganic anions include, but are not limited to, sulfate, phosphate, hydrogen diphosphate, and borate. Examples of polyvalent organic anions include, but are not limited to, water-soluble anions of polycarboxylic acids. In particular, the anion can be an anion of a di- or tri-carboxylic acid, such as oxalic acid, tartaric acid, lactic acid, malic acid, citric acid, aspartic acid, malonic acid, and similar water-soluble polycarboxylic acids optionally containing a hydroxy and/or an amino group. Additional useful polyvalent anions include polycarboxylic amino compounds, for example, polyacrylic acid, ethylenediaminetetraacetic acid (EDTA), ethylenebis(oxyethylenenitrile)tetraacetic acid (EGTA), diethylenetriaminopentaacetic acid (DTPA), N-hydroxyethylethylenediaminetriacetic acid (HEDTA), and mixtures thereof.

In addition, a salt containing a polyvalent metal cation and a polyvalent anion can be used, provided the salt has sufficient water solubility to be dissolved in a solvent for a homogeneous application to surface-crosslinked SAP particles.

The salt can be present in a coating solution together with an optional organic crosslinking agent. The salt typically is present in the coating solution in an amount of about 0.5% to 20%, by weight, for example. The amount of salt present in a coating solution, and the amount applied to the surface-crosslinked polymer particles, is related to the identity of the salt, its solubility in the solvent of the coating solution, the identity of the polyamine applied to the surface-crosslinked polymer particles, and the amount of polyamine applied to the surface-crosslinked polymer particles. In general, the amount of salt applied to the surface-crosslinked polymer particles is sufficient to form a tackless, monolithic polyamine coating and provide coated SAP particles.

In another particular aspect, an organic crosslinking agent can be used in conjunction with the polyamine. In still another aspect, an organic crosslinking agent is applied to the surface crosslinked polymer particles, followed by the polyamine solution. The optional cosolvent can be applied to the surface-crosslinked polymer particles with the organic crosslinking agent, with the polyamine, with both, or alone, either before or after application of the organic crosslinking agent or the polyamine. In either case, the SAP particles then are maintained at a sufficient temperature for a sufficient time to form crosslinks between the polyamine and the crosslinking agent.

In the organic crosslinking process, a multifunctional compound capable of reacting with the amino groups of the polyamine is applied to the surface of the surface-crosslinked polymer particles. The organic crosslinking agent can be the same or different from the surface crosslinking agent. However, as discussed above, the surface crosslinking agent and the crosslinking agent for the polyamine are applied to the base polymer particles during different process steps and the SAP particles are maintained at different temperatures, i.e., the surface crosslinking process utilizes a higher temperature to effect a reaction with the carboxyl groups of the base polymer, and the polyamine crosslinking process utilizes a lower temperature for crosslinking through the nitrogen atoms of the polyamine.

The organic crosslinking process typically utilizes an aqueous solution of the crosslinking agent. The aqueous solution also can contain water-miscible organic solvents, like an alcohol, such as methanol, ethanol, or i-propanol; a polyol, like ethylene glycol or propylene glycol; or acetone.

A solution of an organic crosslinking agent is applied to the surface-crosslinked polymer particles during or after application of the polyamine in an amount to wet predominantly only the outer surfaces of the surface-crosslinked polymer particles. Crosslinking and drying of the coated surface-crosslinked polymer particles then are achieved by maintaining at least the wetted surfaces of the surface-crosslinked polymer particles at a suitable temperature, e.g., about 25° C. to about 100° C., such as about 50° C. to about 100° C., or about 60° C. to about 90° C., for about 5 to about 60 minutes to allow the crosslinking agent to react with the nitrogen atoms of the polyamine.

Typically, the surface-crosslinked polymer particles are treated with a solution of an organic crosslinking agent containing about 0.5% to about 20%, by weight, crosslinking agent, such as about 3% to about 15%, by weight, crosslinking agent in a suitable solvent. The organic crosslinking agent, if present at all, is present in an amount of 0.001% to about 0.5%, by weight of the surface-crosslinked polymer particles, such as 0.001% to about 0.3% by weight. To achieve the full advantage of the present invention, the organic crosslinking agent is present in an amount of about 0.001% to about 0.1%, by weight of the surface-crosslinked polymer particles.

Nonlimiting examples of suitable organic crosslinking agents include, but are not limited to, an alkylene carbonate, such as ethylene carbonate or propylene carbonate; a polyaziridine, such as 2,2-bishydroxymethyl butanol tris[3-(1-aziridine propionate] or bis-N-aziridinomethane; a haloepoxy, such as epichlorohydrin; a polyisocyanate, such as 2,4-toluene diisocyanate; a di- or polyglycidyl compound, such as diglycidyl phosphonates, ethylene glycol diglycidyl ether, or bischlorohydrin ethers of polyalkylene glycols; alkoxysilyl compounds; carbonic acid derivatives, such as urea, thiourea, guanidine, dicyandiamide, 2-oxazolidinone and its derivatives, bisoxazoline, polyoxazolines, di- and polyisocyanates; di- and poly-N-methylol compounds, such as methylenebis(N-methylolmethacrylamide) or melamine-formaldehyde resins; compounds having two or more blocked isocyanate groups, such as trimethylhexamethylene diisocyanate blocked with 2,2,3,6-tetramethylpiperidin-4-one; multifunctional aldehydes, multifunctional ketones, multifunctional acetals, multifunctional ketals, and other organic crosslinking agents known to persons skilled in the art. The organic crosslinking agent can be used alone or in combination.

A solution of the organic crosslinking agent is applied to the surfaces of the surface-crosslinked polymer particles simultaneously with, or before or after, a solution containing the polyamine is applied to the surfaces of the surface-crosslinked polymer particles. The polyamine is applied to the particles after a surface crosslinking step has been completed.

In some aspects, the polyamine solution, and inorganic and/or organic crosslinking agent, are applied to the surface-crosslinked polymer particles in a manner such that each is uniformly distributed on the surfaces of the surface-crosslinked polymer particles. In addition to the crosslinking agent, other optional ingredients can be applied to the surface crosslinked SAP particles in conjunction with the polyamine. Such optional ingredients include, but are not limited to, clay and silica, for example, to impart anticaking properties to the polyamine-coated SAP particles. A clay or silica also can be added to the polyamine-coated SAP particles after application and curing of the polyamine coating.

Any known method for applying a liquid to a solid can be used to apply the polyamine coating to the surface-crosslinked SAP particles, desirably by dispersing a coating solution into fine droplets, for example, by use of a pressurized nozzle or a rotating disc. Uniform coating of the surface-crosslinked polymer particles can be achieved in a high intensity mechanical mixer or a fluidized mixer which suspends the surface-crosslinked polymer particles in a turbulent gas stream. Methods for the dispersion of a liquid onto the surfaces of surface-crosslinked polymer particles are known in the art, see, for example, U.S. Pat. No. 4,734,478, incorporated herein by reference in a manner that is consistent herewith.

In some aspects, methods of coating the surface-crosslinked polymer particles include applying the polyamine and crosslinking agent simultaneously. When an inorganic salt is used as a crosslinking agent, the polyamine and salt desirably are applied via two separate nozzles to avoid an interaction before application to the surfaces of the surface-crosslinked polymer particles. A desirable method of coating the surface-crosslinked polymer is a sequential addition of the components. A more desirable method is a first application of the polyamine, followed by an application of the crosslinking agent.

The resulting polyamine coated surface-crosslinked polymer particles then are maintained at about 25° C. to about 100° C., such as about 30° C. to about 80° C., or about 35° C. to about 60° C., for a sufficient time to maintain a hydrophobic particle surface, e.g., about 5 to about 60 minutes. In particular, the polyamine coating typically is applied to surface-crosslinked SAP particles that have not completely cooled after the surface-crosslinking process. Accordingly, the polyamine-coating step utilizes the latent heat of the surface-crosslinked SAP particles. If necessary, external heat can be applied to maintain a desired particle temperature up to about 100° C. and cure the polyamine coating. The temperature of the polyamine-coated SAP particles is maintained at about 100° C. or less to avoid, or at least minimize, reactions that form covalent bonds between the polyamine coating and the carboxyl groups of the base polymer.

After application of the polyamine, water, optional cosolvent, and optional crosslinking agent to the surface-crosslinked SAP particles, the coated SAP particles are mixed at about 25° C. to about 100° C., e.g., 25, 30, 35, 40, 45, 50, 55, 60, 65, 70, 75, 80, 85, 90, 95, or 100° C., for about 5 to about 60 minutes in a paddle mixer, for example, such as those available from Ruberg-Mischtechnik AG, Nieheim, Germany and Nara Machining Co., Ltd., Frechen, Germany. Other suitable mixers include Patterson-Kelly mixers, DRAIS turbulence mixers, Lödige mixers, Schugi mixers, screw mixers, and pan mixers. After mixing, a polyamine coated SAP of the present invention results, i.e., a polyamine coating, wherein covalent bonds between the polyamine and the carboxyl groups of the base polymer are minimized.

The polyamine-coated SAP particles of the present invention have excellent absorption properties, permeability, and gel integrity. In some particular aspects, the SAP particles can have a centrifuge retention capacity of at least 25 g/g. In other particular aspects, the SAP particles can have a wicking index of less than 2.3 cm after 1 minute, less than 3 cm after 5 minutes, and less than 6.5 cm after 10 minutes. In still other aspects, the present particles can also exhibit a gel integrity of at least 2.5, desirably at least 3, more desirably at least 3.5, and most desirably at least 4.0. In yet other aspects, the present SAP particles further exhibit a free swell gel bed permeability of at least 200, desirably at least 210, 220, 230, 240, or 250, and more desirably 260, 270, 280, 290, or 300 Darcies and desirably a gel bed permeability (0.3 psi) of at least 3, more desirably at least 4, 5, 6, or 7, and most desirably at least 8, 9, or 10 Darcies. In still other aspects, the present SAP particles can exhibit a free swell gel bed permeability (FSGBP) that is at least two times greater than the FSGBP of identical surface-crosslinked SAP particles free of a polyamine coating.

The present invention, therefore, provides polyamine-coated SAP particles having improved absorbency, fluid permeability, and gel integrity. Surprisingly, the absorbency, fluid permeability, and gel integrity properties are independent of wicking index, i.e., as wicking index decrease, the expected decrease in the absorbency and permeability properties are not observed.

In some aspects, the particles can have a transient hydrophobic surface, as measured by a wicking index. The polyamine-coated SAP particles of the present invention, therefore, can exhibit a delayed swelling mechanism upon insult with urine, which overcomes the problem of gel blocking. The final result is a more complete utilization of the full absorbing capabilities of the SAP particles.

In particular, the SAP particles of the present invention, after contact with a 0.9% saline solution, exhibit a delay of at least 5 seconds prior to absorbing the saline solution. Desirably, the SAP particles exhibit a delay of at least 10 seconds, such as at least 15 seconds, prior to absorbing a 0.9% saline solution that contacts the SAP particle. In most desirable aspects, the SAP particles exhibit at least a 20 second delay prior to absorbing a 0.9% saline solution that contacts the particles.

The present invention also provides polyamine-coated SAP particles that have a hydrophobic surface when a cosolvent is applied as a component of the coating solution, which reduces SAP particle agglomeration attributed to the viscous, tacky nature of polyamines. The present invention provides polyamine-coated SAP particles having a hydrophobic surface when the SAP particles are maintained at a relatively low temperature, i.e., about 25° C. to about 100° C., desirably about 50° C. to about 100° C., and most desirably about 60° C. to about 80° C., for about 5 to about 60 minutes, after application of the polyamine coating. The present invention also provides polyamine-coated SAP particles having a hydrophilic surface when an inorganic or organic crosslinking agent is applied as a component of the coating solution, and the SAP particles are maintained at a relatively low temperature, i.e., about 25° C. to about 100° C., desirably about 50° C. to about 100° C., and most desirably about 60° C. to about 80° C., for about 5 to about 60 minutes, after application of the polyamine coating.

In some aspects, a polyamine is applied to surface-crosslinked SAP particles in a manner such that the polyamine and any optional crosslinking agent are uniformly distributed on the surfaces of the surface-crosslinked SAP particles. The resulting coated surface-crosslinked SAP particles then are maintained at about 25° C. to about 100° C., such as about 50° C. to about 100° C., or about 60° C. to about 80° C., for sufficient time, e.g., about 5 to about 60 minutes, such as about 10 to about 30 minutes, to cure the polyamine coating, while minimizing covalent crosslinks between the polyamine coating and the carboxyl groups of the base polymer.

To demonstrate the unexpected advantages, including but not limited to a transient surface hydrophobicity, provided by the coated SAP particles of some aspects of the present invention, polyamine-coated SAP particles were prepared and tested for centrifuge retention capacity (CRC, expressed in g/g), absorbency under load (AUL 0.9 psi, expressed in g/g), free swell gel bed permeability (FSGBP, expressed in Darcies), gel bed permeability (GBP 0.3 psi, expressed in Darcies), gel integrity (GI) (scale of 1 to 4), and fluid wicking index (cm/min). These tests were performed using the procedures described above.

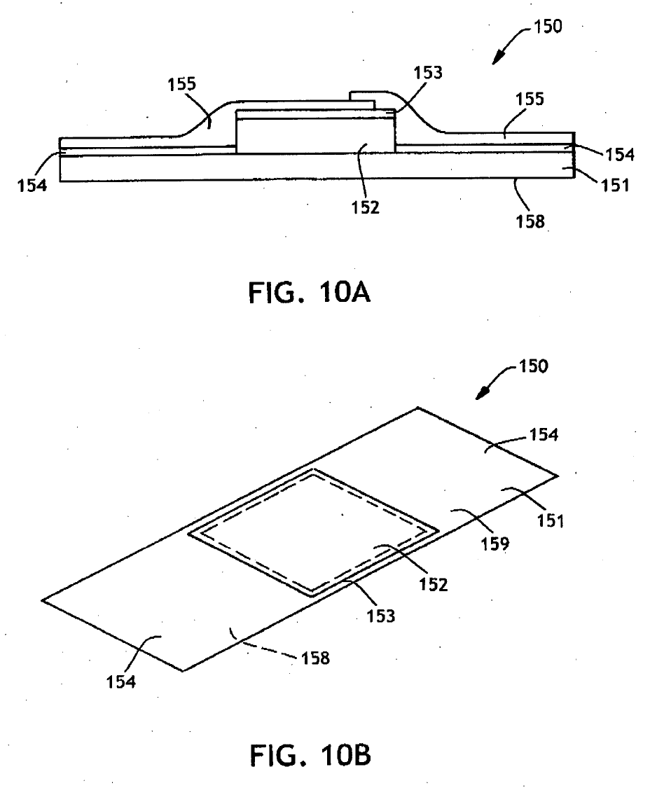

In addition to the absorbent article described above, the present invention may be exemplified as an absorbent bandage. Attention is directed to FIGS. 10A and 10B, which show a possible configuration for a bandage of the present invention. FIG. 10A shows a cross-section view of the absorbent bandage with optional layers described below. FIG. 10B shows a perspective view of the bandage of the present invention with some of the optional or removable layers not being shown. The absorbent bandage 150 has a strip 151 of material having a body-facing side 159 and a second side 158 which is opposite the body-facing side. The strip is essentially a backsheet and is desirably prepared from the same materials described above for the backsheet. In addition, the strip may be an apertured material, such as an apertured film, or material which is otherwise gas permeable, such as a gas permeable film. The strip 151 supports an absorbent core 152 comprising SAP particles of the present invention which is attached to the body-facing side 159 of the strip. In addition, an absorbent protective layer 153 may be applied to the absorbent core 152 and can be coextensive with the strip 151.

The absorbent bandage 150 of the present invention may also have a pressure sensitive adhesive 154 applied to the body-facing side 159 of the strip 151. Any pressure sensitive adhesive may be used, provided that the pressure sensitive adhesive does not irritate the skin of the user. Suitably, the pressure sensitive adhesive is a conventional pressure sensitive adhesive which is currently used on similar conventional bandages. This pressure sensitive adhesive is desirably not placed on the absorbent core 152 or on the absorbent protective layer 153 in the area of the absorbent core 152. If the absorbent protective layer is coextensive with the strip 151, then the adhesive may be applied to areas of the absorbent protective layer 153 where the absorbent core 152 is not located. By having the pressure sensitive adhesive on the strip 151, the bandage is allowed to be secured to the skin of a user in need of the bandage. To protect the pressure sensitive adhesive and the absorbent, a release strip 155 can be placed on the body-facing side 159 of the bandage. The release liner may be removably secured to the article attachment adhesive and serves to prevent premature contamination of the adhesive before the absorbent article is secured to, for example, the skin. The release liner may be placed on the body-facing side of the bandage in a single piece (not shown) or in multiple pieces, as is shown in FIG. 10A.

In another aspect of the present invention, the absorbent core of the bandage may be placed between a folded strip. If this method is used to form the bandage, the strip is suitably fluid permeable.

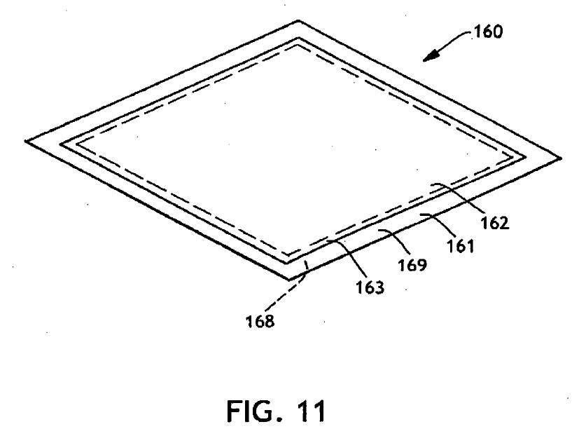

Absorbent furniture and/or bed pads or liners are also included within the present invention. As is shown in FIG. 11, a furniture or bed pad or liner 160 (hereinafter referred to as a “pad”) is shown in perspective. The pad 160 has a fluid impermeable backsheet 161 having a furniture-facing side or surface 168 and an upward facing side or surface 169 which is opposite the furniture-facing side or surface 168. The fluid impermeable backsheet 161 supports the absorbent core 162 which comprises SAPs of the present invention, and which is attached to the upward facing side 169 of the fluid impermeable backsheet. In addition, an optional absorbent protective layer 163 may be applied to the absorbent core. The optional substrate layer of the absorbent core can be the fluid impermeable layer 161 or the absorbent protective layer 163 of the pad.

To hold the pad in place, the furniture-facing side 168 of the pad may contain a pressure sensitive adhesive, a high friction coating or other suitable material which will aid in keeping the pad in place during use. The pad of the present invention can be used in a wide variety of applications including placement on chairs, sofas, beds, car seats and the like to absorb any fluid which may come into contact with the pad.

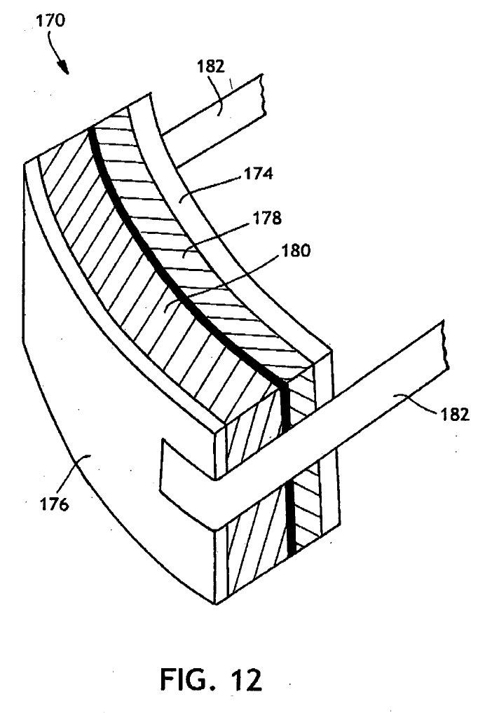

Sports or construction accessories, such as an absorbent headband for absorbing perspiration or drying off equipment are also included within the present invention. As is shown in FIG. 12, an absorbent sweatband 170 is shown in perspective. The sweatband 170 has an absorbent core 180 disposed between an optional topsheet 174 and/or an optional fluid impervious backsheet 176. The absorbent core 180 comprises the SAP particles of the present invention, and in some aspects can include an optional additional region 178 (such as a distribution layer), if desired. The sweatband can be useful to intercept perspiration prior to contact with the hands or eyes. VELCRO or other fastening device 182 can be used to facilitate adjustment or comfort.

The present invention may be better understood with reference to the following examples.

### EXAMPLES

### Example 1

Surface-crosslinked polymer particles, HySorb B-8700AD available from BASF AG, Ludwigshafen, Germany, were preheated in a laboratory oven set at a predetermined coating temperature. When the polymer particles (1 kg) attained a predetermined coating temperature, the surface-crosslinked polymer particles were transferred to a preheated laboratory Lödige mixer. The particles were maintained at the constant predetermined temperature throughout the coating step. Addition of a polyvinylamine coating solution (i.e., 40 grams LUPAMIN 9095 and 15 grams of deionized water) to the preheated polymer particles was performed by a disposable syringe, dropwise over 5 minutes at a Lödige mixing speed of 449 rpm. After complete addition of the coating solution, the Lödige mixing speed was reduced to 79 rpm, and mixing was continued for 30 minutes. The results can be seen in Tables 1-3.

TABLE 1

| | | Propylene | | | |
|---|---|---|---|---|---|
| | | glycol | | | Residence |
| | LUPAMIN¹⁾ | (PG) | H₂O | Coating | Time |
| Example | 9095 (wt %) | (wt %) | (wt %) | Temp (° C.) | (min.) |
| Control | Base polymer (HySorb B-8700AD) | | | | |
| 1a | 4.0 | 0.0 | 1.5 | 60 | 30 |
| 1b | 4.0 | 0.0 | 1.5 | 70 | 30 |
| 1c | 4.0 | 0.0 | 1.5 | 80 | 30 |

¹⁾LUPAMIN 9095, available from BASF Corporation, Florham Park, New Jersey, U.S.A., contains 5–10% linear polyvinylamine, average molecular weight 340,000.

TABLE 2

| | | | | | | Gel | |
|---|---|---|---|---|---|---|---|
| | | | AUL | | | Integrity | |
| | PSD | CRC | 0.9 psi | FS GBP | GBP 0.3 psi | (1~4 | SAP |
| Example | (>860μ wt %) | (g/g) | (g/g) | (Darcies) | (Darcies) | scale) | Surface |
| Control | | 26.28 | 19.60 | 85.5 | 8.48 | 1.0 | Hydrophilic |
| 1a | 5.94 | 24.90 | 17.23 | 229.0 | 6.59 | 4.0 | Hydrophilic |
| 1b | 6.27 | 25.29 | 18.91 | 181.4 | 7.29 | 4.0 | Hydrophilic |
| 1c | 6.66 | 25.66 | 19.25 | 152.8 | 6.38 | 2.5 | Hydrophilic |

TABLE 3
Wicking index (cm)

| Example | 1 min. | 5 min. | 10 min. |
|---|---|---|---|
| Control | 6.0 | 11.0 | 15.0 |
| 1a | 2.6 | 5.5 | 7.0 |
| 1b | 3.5 | 6.0 | 8.0 |
| 1c | 3.0 | 6.0 | 7.0 |

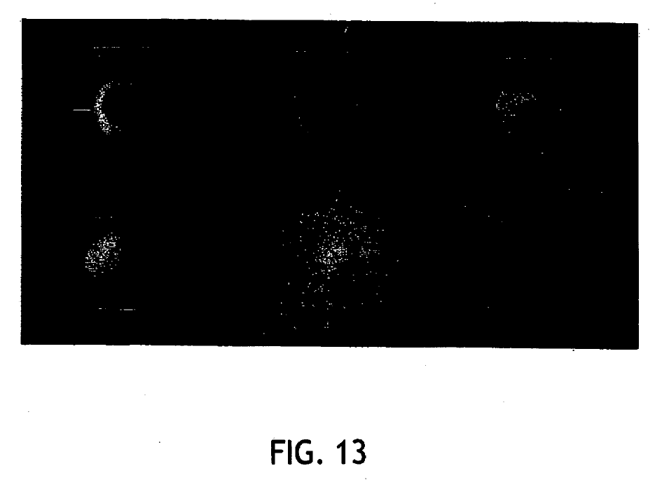

The SAP particles of Example 1 have a hydrophilic surface because a cosolvent was not included in the polyamine coating process. FIG. 13 contains photographs showing the addition of 0.9% saline fluid added to the hydrophilic SAP particles of Example 1. The sequence of photographs demonstrates the addition of saline to the SAP particles and the results 30 seconds after the saline addition. Note that the wetted particles in the final photograph have a poor gel integrity.

### Example 2

Surface-crosslinked polymer particles, HySorb B-8700AD, were preheated in a laboratory oven set at a predetermined coating temperature. When the polymer particles (1 kg) attained a predetermined coating temperature, the particles were transferred to a preheated laboratory Lödige mixer. The polymer particles were maintained at the constant predetermined temperature throughout the coating step. Addition of a polyvinylamine coating solution (40 grams LUPAMIN 9095, 10 grams propylene glycol (PG), and 15 grams of deionized (DI) water) to the preheated polymer particles was performed by disposable syringe, dropwise over 5 minutes at a Lödige mixing speed of 449 rpm. After complete addition of the coating solution, the Lödige mixing speed was reduced to 79 rpm, and mixing was continued for 30 minutes. The results can be seen in Tables 4-6.

TABLE 4

| | | Propylene | | | |
|---|---|---|---|---|---|
| | LUPAMIN | glycol | | | Residence |
| | 9095 | (PG) | H₂O | Coating | Time |
| Example | (wt %) | (wt %) | (wt %) | Temp (° C.) | (min.) |
| Control | Base polymer (HySorb B-8700AD) | | | | |
| 2a | 4.0 | 1.0 | 1.5 | 60 | 30 |
| 2b | 4.0 | 1.0 | 1.5 | 70 | 30 |
| 2c | 4.0 | 1.0 | 1.5 | 80 | 30 |

TABLE 5

| | | | | | | Gel | |
|---|---|---|---|---|---|---|---|
| | | | AUL | | | Integrity | |
| | PSD | CRC | 0.9 psi | FS GBP | GBP 0.3 psi | (1~4 | |
| Example | (>860μ wt %) | (g/g) | (g/g) | (Darcies) | (Darcies) | scale) | SAP Surface |
| Control | | 26.28 | 19.60 | 85.5 | 8.48 | 1.0 | Hydrophilic |
| 2a | 0.22 | 24.59 | 17.45 | 342.5 | 2.45 | 4.0 | Hydrophobic |
| 2b | 0.24 | 25.14 | 17.04 | 356.1 | 3.16 | 4.0 | Hydrophobic |
| 2c | 0.34 | 25.69 | 17.39 | 303.6 | 3.34 | 3.5 | Hydrophobic |

TABLE 6
Wicking index (cm)

| Example | 1 min. | 5 min. | 10 min. |
|---|---|---|---|
| Control | 6.0 | 11.0 | 15.0 |
| 2a | 0.4 | 1.0 | 1.5 |
| 2b | 0.4 | 1.0 | 1.5 |
| 2c | 0.4 | 1.0 | 2.0 |

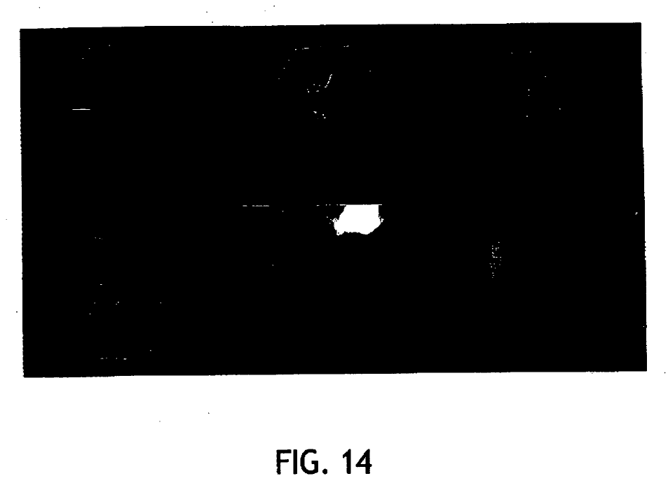

The photographs in FIG. 14 show the improved gel integrity achieved by the polyamine-coated SAP particles of one particular aspect of the present invention which have a transient hydrophobic surface.

### Example 3

Surface-crosslinked polymer particles, HySorb B-8700AD, were preheated in a laboratory oven set at a predetermined temperature. When the polymer particles (1 kg) attained a predetermined temperature, the particles were transferred to a preheated laboratory Lödige mixer. The polymer particles were maintained at the constant predetermined temperature throughout the coating step. Two solutions were prepared. Preparation of Solution 1: alum solution (35.8 grams, 28.1 wt % aluminum sulfate) was placed in first disposable syringe, and Solution 2: polyvinylamine coating solution (40 or 20 grams LUPAMIN 9095, 10 grams PG) was placed in second disposable syringe. Solution 1 was added first, then Solution 2, to the preheated polymer particles. The additions were performed dropwise over 5 minutes at a Lödige mixing speed of 449 rpm. After complete addition of the coating solutions, the Lödige mixing speed was reduced to 79 rpm, and mixing was continued for 30 minutes. The results can be seen in Tables 7-9.

TABLE 7

| | | Propylene | | | |
|---|---|---|---|---|---|
| | LUPAMIN | glycol | Aluminum | Coating | Residence |
| | 9095 | (PG) | sulfate | Temp | Time |
| Example | (wt %) | (wt %) | (wt %) | (° C.) | (min.) |
| Control | Base polymer (HySorb B-8700AD) | | | | |
| 3a | 4.0 | 1.0 | — | 60 | 30 |
| 3b | 4.0 | 1.0 | 1 | 60 | 30 |
| 3c | 4.0 | 1.0 | 1 | 100 | 30 |
| 3d | 2.0 | 1.0 | — | 60 | 30 |
| 3e | 2.0 | 1.0 | 1 | 60 | 30 |
| 3f | 2.0 | 1.0 | 1 | 100 | 30 |

TABLE 8

| | | | | | | Gel | |
|---|---|---|---|---|---|---|---|
| | | | AUL | | | Integrity | |
| | PSD | CRC | 0.9 psi | FS GBP | GBP 0.3 psi | (1~4 | |
| Example | (>860μ wt %) | (g/g) | (g/g) | (Darcies) | (Darcies) | scale) | SAP Surface |
| Control | | 26.28 | 19.60 | 85.53 | 8.58 | 1.0 | Hydrophilic |
| 3a | 0.17 | 24.89 | 18.50 | 351.60 | 2.06 | 4.0 | Hydrophobic |
| 3b | 0.19 | 24.30 | 16.94 | 317.48 | 7.01 | 4.0 | Hydrophilic |
| 3c | 0.40 | 24.93 | 16.79 | 323.23 | 3.63 | 4.0 | Hydrophilic |
| 3d | 1.94 | 23.85 | 18.11 | 253.42 | 11.50 | 3.5 | Hydrophobic |
| 3e | 0.14 | 24.80 | 17.75 | 247.73 | 9.20 | 2.5 | Hydrophilic |
| 3f | 0.64 | 25.00 | 16.97 | 260.73 | 9.55 | 3.5 | Hydrophilic |

TABLE 9
Wicking index (cm)

| Example | 1 min. | 5 min. | 10 min. |
|---|---|---|---|
| Control | 7.0 | 11.0 | 14.5 |
| 3a | 1.0 | 1.5 | 2.0 |
| 3b | 5.5 | 7.5 | 9.0 |
| 3c | 3.0 | 4.9 | 6.0 |
| 3d | 1.0 | 4.0 | 6.0 |
| 3e | 7.0 | 10.5 | 13.0 |
| 3f | 5.5 | 9.5 | 11.0 |

### Example 4

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The polymer particles were maintained at 60° C. throughout the coating step. Two solutions were prepared. Preparation of Solution 1: ionic crosslinker solution (35.8 grams of alum solution or 40 grams of 25% aqueous sodium sulfate solution or 37 grams of 27% aqueous sodium silicate solution or 9.26 grams of a 25% aqueous solution of trisodium phosphate) was placed in a first disposable syringe and Solution 2: polyvinylamine coating solution (20 grams LUPAMIN 9095, 10 grams PG) was placed in second disposable syringe. Solution 1 was added first, then Solution 2, to the preheated polymer particles. The additions were dropwise over 5 minutes at a Lödige mixing speed of 449 rpm. After complete addition of the coating solutions, the Lödige mixing speed was reduced to 79 rpm, and mixing was continued for 30 minutes. The results can be seen in Tables 10-12.

TABLE 10

| | | Propylene | | | |
|---|---|---|---|---|---|
| | | glycol | Ionic | Coating | Residence |
| | LUPAMIN | (PG) | crosslinker | Temp | Time |
| Example | 9095 wt % | (wt %) | (wt %) | (° C.) | (min.) |
| Control | Base polymer (HySorb B-8700AD) | | | | |
| 4a | 2.0 | 1.0 | 1% | 60 | 30 |
| | | | Al₂(SO₄)₃ | | |
| 4b | 2.0 | 1.0 | 1% Na | 60 | 30 |
| | | | Silicate | | |
| 4c | 2.0 | 1.0 | 1% | 60 | 30 |
| | | | Na₂SO₄ | | |
| 4d | 2.0 | 1.0 | 0.1% | 60 | 30 |
| | | | Na₃PO₄ | | |
| 4e | 2.0 | 1.0 | 0.2% | 60 | 30 |
| | | | Na₃PO₄ | | |

TABLE 11

| | | | | | | Gel | |
|---|---|---|---|---|---|---|---|
| | | | AUL | | | intergrity | |
| | PSD | CRC | 0.9 psi | FS GBP | GBP 0.3 psi | (1~4 | SAP |
| Example | (>860μ wt %) | (g/g) | (g/g) | (Darcies) | (Darcies) | scale) | Surface |
| Control | | 26.28 | 19.60 | 85.53 | 8.58 | 1.0 | Hydrophilic |
| 4a | 0.14 | 24.80 | 17.75 | 247.73 | 9.20 | 2.5 | Hydrophilic |
| 4b | 6.86 | 25.58 | 18.95 | 308.50 | 8.64 | 4.0 | Hydrophilic |
| 4c | 0.07 | 25.08 | 18.25 | 291.20 | 7.97 | 4.0 | Hydrophilic |
| 4d | 0.10 | 24.91 | 18.35 | 302.45 | 8.36 | 4.0 | Hydrophilic |
| 4e | 0.67 | 25.21 | 17.62 | 298.66 | 6.72 | 4.0 | Hydrophilic |

TABLE 12
Wicking index (cm)

| Example | 1 min. | 5 min. | 10 min. |
|---|---|---|---|
| Control | 7.0 | 11.0 | 14.5 |
| 4a | 7.0 | 10.5 | 13.0 |
| 4b | 1.5 | 3.0 | 4.2 |
| 4c | 1.0 | 2.5 | 4.3 |
| 4d | 5.5 | 9.0 | 10.5 |
| 4e | 6.2 | 9.5 | 11.0 |

### Example 5

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The polymer particles were maintained at 60° C. throughout the coating step. Two solutions were prepared. Preparation of Solution 1: ionic crosslinker solution (varied grams of alum solution) was placed in a first disposable syringe and Solution 2: polyvinylamine coating solution (40 or 20 grams LUPAMIN 9095, 10 grams PG) was placed in a second disposable syringe. Solution 1 was added first, followed by Solution 2, to the preheated polymer particles. The additions were dropwise over 5 minutes at a Lödige mixing speed of 449 rpm. After complete addition of the coating solution, the Lödige mixing speed was reduced to 79 rpm, and mixing was continued for 30 minutes. The results can be seen in Tables 13-15.

TABLE 13

| | | Propylene | | | |
|---|---|---|---|---|---|
| | LUPAMIN | glycol | Aluminum | Coating | Residence |
| | 9095 | (PG) | sulfate | Temp | Time |
| Example | (wt %) | (wt %) | (wt %) | (° C.) | (min.) |
| Control | Base polymer (HySorb B-8700AD) | | | | |
| 5a | 2.0 | 1.0 | 1.0 | 60 | 30 |
| 5b | 2.0 | 1.0 | 0.7 | 60 | 30 |
| 5c | 2.0 | 1.0 | 0.5 | 60 | 30 |
| 5d | 2.0 | 1.0 | 0.3 | 60 | 30 |

TABLE 14

| | | | | | | Gel | |
|---|---|---|---|---|---|---|---|
| | | | AUL | | | integrity | |
| | PSD | CRC | 0.9 psi | FS GBP | GBP 0.3 psi | (1~4 | SAP |
| Example | (>860μ wt %) | (g/g) | (g/g) | (Darcies) | (Darcies) | scale) | Surface |
| Control | | 26.28 | 19.60 | 85.53 | 8.58 | 1.0 | Hydrophilic |
| 5a | 0.14 | 24.80 | 17.75 | 247.7 | 9.20 | 2.5 | Hydrophilic |
| 5b | 0.14 | 24.98 | 18.27 | 251.2 | 9.70 | 4.0 | Hydrophilic |
| 5c | 0.13 | 25.05 | 18.16 | 285.2 | 8.96 | 4.0 | Hydrophilic |
| 5d | 0.14 | 25.64 | 18.67 | 291.1 | 8.79 | 4.0 | Hydrophilic |

TABLE 15
Wicking index (cm)

| Example | 1 min. | 5 min. | 10 min. |
|---|---|---|---|
| Control | 7.0 | 11.0 | 14.5 |
| 5a | 8.0 | 12.0 | 15.0 |
| 5b | 4.0 | 9.0 | 10.0 |
| 5c | 5.5 | 7.0 | 8.5 |
| 5d | 5.5 | 8.0 | 10.0 |

### Example 6

The procedure of Example 5 was repeated to show the absorbency, gel permeability, and gel integrity of HySorb B-8700AD particles coated with LUPAMIN 9095, propylene glycol, and aluminum sulfate solution. The results can be seen in Tables 16-18.

TABLE 16

| | | Propylene | | | |
|---|---|---|---|---|---|
| | LUPAMIN | glycol | Aluminum | Coating | Residence |
| | 9095 | (PG) | sulfate | Temp | Time |
| Example | (wt %) | (wt %) | (wt %) | (° C.) | (min.) |
| Control | Base polymer (HySorb B-8700AD) | | | | |
| 6a | 1.0 | 1.0 | 0.2 | 60 | 30 |
| 6b | 3.0 | 1.0 | 0.2 | 60 | 30 |
| 6c | 1.0 | 1.0 | 0.8 | 60 | 30 |
| 6d | 3.0 | 1.0 | 0.8 | 60 | 30 |
| 6e | 1.0 | 1.0 | 0.2 | 80 | 30 |
| 6f | 3.0 | 1.0 | 0.2 | 80 | 30 |
| 6g | 1.0 | 1.0 | 0.8 | 80 | 30 |
| 6h | 3.0 | 1.0 | 0.8 | 80 | 30 |
| 6i | 2.0 | 1.0 | 0.5 | 70 | 30 |
| 6j | 2.0 | 1.0 | 0.5 | 70 | 30 |

TABLE 17

| | | | | | | Gel | |
|---|---|---|---|---|---|---|---|
| | | | AUL | | | integrity | |
| | PSD | CRC | 0.9 psi | FS GBP | GBP 0.3 psi | (1~4 | |
| Example | (>860μ wt %) | (g/g) | (g/g) | (Darcies) | (Darcies) | scale) | SAP Surface |
| Control | | 26.28 | 19.60 | 85.53 | 8.58 | 1.0 | Hydrophilic |
| 6a | 0.08 | 24.92 | 18.88 | 214.2 | 14.05 | 3.0 | Hydrophobic |
| 6b | 0.07 | 25.81 | 18.29 | 298.0 | 4.61 | 4.0 | Hydrophilic |
| 6c | 0.07 | 25.39 | 20.10 | 206.9 | 10.93 | 1.0 | Hydrophilic |
| 6d | 0.02 | 24.79 | 19.77 | 273.4 | 8.12 | 4.0 | Hydrophobic |
| 6e | 0.31 | 26.23 | 21.26 | 225.5 | 10.65 | 3.0 | Hydrophilic |
| 6f | 0.22 | 25.54 | 20.02 | 298.5 | 5.92 | 4.0 | Hydrophilic |
| 6g | 0.36 | 25.40 | 20.07 | 213.7 | 10.08 | 1.5 | Hydrophilic |
| 6h | 0.28 | 25.09 | 19.04 | 293.1 | 6.30 | 4.0 | Hydrophilic |
| 6i | 0.14 | 25.50 | 20.08 | 251.2 | 9.18 | 4.0 | Hydrophilic |
| 6j | 0.14 | 25.44 | 19.86 | 282.0 | 9.63 | 4.0 | Hydrophilic |

TABLE 18
Wicking index (cm)

| Example | 1 min. | 5 min. | 10 min. |
|---|---|---|---|
| Control | 7.0 | 11.0 | 14.5 |
| 6a | 5.5 | 9.0 | 10.0 |
| 6b | 2.0 | 4.0 | 5.6 |
| 6c | 8.0 | 12.0 | 14.5 |
| 6d | 5.0 | 8.0 | 9.0 |
| 6e | 5.6 | 7.0 | 8.0 |
| 6f | 1.8 | 4.0 | 6.0 |
| 6g | 9.2 | 14.0 | 15.0 |
| 6h | 5.9 | 7.5 | 8.5 |
| 6i | 6.5 | 8.2 | 10.5 |
| 6j | 6.5 | 9.2 | 10.5 |

### Example 7

Surface-crosslinked polymer particles, HySorb B-8700AD, were preheated in a laboratory oven at 80° C. When the polymer particles (1 kg) reached 80° C., the particles were transferred to a preheated (80° C.) laboratory Lödige mixer. The polymer particles were maintained at 80° C. throughout the coating step. Two solutions were prepared. Preparation of Solution 1: covalent crosslinker solution (1 or 2 or 3 grams of ethylene glycol diglycidyl ether (EGDGE) in 15 grams of DI water) was placed in a first disposable syringe and solution 2: polyvinylamine coating solution (40 grams LUPAMIN 9095, 10 grams PG) was placed in a second disposable syringe. Solution 1 was added first, then Solution 2, to the preheated polymer particles dropwise. The additions were over 5 minutes at a Lödige mixing speed of 449 rpm. After complete addition of the coating solution, the Lödige mixing speed was reduced to 79 rpm, and mixing was maintained for 30 minutes. The results can be seen in Tables 19-21.

TABLE 19

| | | | | | |
|---|---|---|---|---|---|
| | | Propylene | | | |
| | LUPAMIN | glycol | | | |
| | 9095 | (PG) | EGDGE | H₂O | Coating |
| Example | (wt %) | (wt %) | (wt %) | (wt %) | Temp (° C.) |

| Control | Base polymer (HySorb B-8700AD) | | | | |
|---|---|---|---|---|---|
| 7a | 4.0 | 1.0 | 0.1 | 1.5 | 80 |
| 7b | 4.0 | 1.0 | 0.2 | 1.5 | 80 |
| 7c | 4.0 | 1.0 | 0.3 | 1.5 | 80 |

TABLE 20

| | | | | | | Gel | |
|---|---|---|---|---|---|---|---|
| | | | AUL | | | integrity | |
| | PSD | CRC | 0.9 psi | FS GBP | GBP 0.3 psi | (1~4 | SAP |
| Example | (>860μ wt %) | (g/g) | (g/g) | (Darcies) | (Darcies) | scale) | Surface |
| Control | | 26.28 | 19.60 | 85.5 | 8.48 | 1.0 | Hydrophilic |
| 7a | 0.31 | 25.42 | 18.05 | 254.65 | 10.83 | 3.5 | Hydrophilic |
| 7b | 0.54 | 24.72 | 18.05 | 231.31 | 9.69 | 3.0 | Hydrophilic |
| 7c | 0.31 | 24.60 | 18.60 | 188.08 | 9.52 | 2.5 | Hydrophilic |

TABLE 21
Wicking index (cm)

| Example | 1 min. | 5 min. | 10 min. |
|---|---|---|---|
| Control | 7.0 | 11.0 | 14.5 |
| 7a | 4.0 | 5.5 | 6.5 |
| 7b | 5.0 | 7.0 | 8.0 |
| 7c | 6.5 | 9.0 | 10.0 |

### Example 8

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The particles were maintained at a constant 60° C. throughout the coating step. Addition of polyvinylamine coating solution (40 grams LUPAMIN 9095, 10 grams cosolvent, and 15 grams of DI water) to the preheated polymer particles was performed using a disposable syringe. The addition was dropwise over 5 minutes at a Lödige mixing speed of 449 rpm. After complete addition of the coating solution, the Lödige mixing speed was reduced to 79 rpm, and mixing was continued for 30 minutes. The cosolvents used in this example were: propylene glycol (PG), 1,3-propanediol (PDO), isopropyl alcohol (IPA), methanol (MeOH), and ethylene glycol (EG). The results can be seen in Tables 22-24.

TABLE 22

| | LUPAMIN | | | | Residence |
|---|---|---|---|---|---|
| | 9095 | Cosolvent | H₂O | Coating | Time |
| Example | (wt %) | (wt %) | (wt %) | Temp (° C.) | (min.) |
| 8a | 4.0 | 1% PG | 1.5 | 60 | 30 |
| 8b | 4.0 | 1% PDO | 1.5 | 60 | 30 |
| 8c | 4.0 | 1% IPA | 1.5 | 60 | 30 |
| 8d | 4.0 | 1% MeOH | 1.5 | 60 | 30 |
| 8e | 4.0 | 1% EG | 1.5 | 60 | 30 |

TABLE 23

| | | | | | | Gel | |
|---|---|---|---|---|---|---|---|
| | | | AUL | | | integrity | |
| | PSD | CRC | 0.9 psi | FS GBP | GBP 0.3 psi | (1~4 | |
| Example | (>860μ wt %) | (g/g) | (g/g) | (Darcies) | (Darcies) | scale) | SAP Surface |
| 8a | 0.22 | 24.59 | 16.90 | 342.5 | 2.45 | 4.0 | Hydrophobic |
| 8b | 0.23 | 24.65 | 16.39 | 286.5 | 3.76 | 4.0 | Hydrophobic |
| 8c | 0.38 | 23.30 | 16.62 | 289.8 | 3.53 | 4.0 | Hydrophobic |
| 8d | 0.33 | 24.55 | 17.50 | 302.6 | 3.83 | 4.0 | Hydrophobic |
| 8e | 0.70 | 24.64 | 16.60 | 328.9 | 3.00 | 4.0 | Hydrophobic |

TABLE 24
Wicking index (cm)

| Example | 1 min. | 5 min. | 10 min. |
|---|---|---|---|
| 8a | 0.4 | 1.0 | 1.5 |
| 8b | 0.8 | 1.0 | 2.0 |
| 8c | 0.5 | 1.5 | 2.5 |
| 8d | 1.8 | 2.5 | 3.0 |
| 8e | 0.5 | 1.4 | 2.0 |

Examples 1 through 8 show that polyamine-coated SAP particles of the present invention demonstrate excellent permeability (0.3 psi GBP), absorbency is maintained (CRC), and gel integrity (GI) is improved, in addition to a reduced agglomeration of particles when the SAP particle surface is rendered hydrophobic by incorporating a cosolvent in the coating process.

Furthermore, an additional unexpected result is observed with respect to wicking index. Typically, as the wicking index of a SAP particle decreases, the permeability of the SAP particle also decreases. This is attributed to an increase in gel blocking associated with a low wicking index. In contrast, the present polyamine-coated SAP particles do not exhibit a decrease in permeability properties, as demonstrated in the Examples, even though the wicking index of the polyamine-coated particles may be lower than the wicking index of a control polymer. In some aspects, a decrease in wicking index resulted in an increase in permeability properties. Accordingly, the improved absorbency, permeability, and gel integrity properties of the present polyamine-coated SAP particles are independent of the wicking index demonstrated by the particles.

The balanced properties of absorbency, permeability, and gel integrity demonstrated by the present polyamine-coated SAP particles, and the essential independence of these properties from the wicking index, can be attributed to the relatively low temperature at which the surface-crosslinked SAP particles are maintained after application of the polyamine to the surfaces of the surface-crosslinked SAP particles. In particular, the low curing temperatures maintain an excellent gel integrity, which is adversely affected by a typically high temperature cure of the polyamine coating.

### Example 9

Surface-crosslinked polymer particles, HySorb B-8700AD, were preheated in a laboratory oven at 60° C. When the polymer particles (1 kg) reached 60° C., the particles were transferred to a preheated (60° C.) laboratory Lödige mixer. The particles were maintained at a constant 60° C. throughout the coating step. Addition of polyvinylamine coating solution (40 grams LUPAMIN 9095, 10 grams cosolvent, and 15 grams of DI water) to the preheated polymer particles was performed using a disposable syringe. The addition was dropwise over 5 minutes at a Lödige mixing speed of 449 rpm. After complete addition of the coating solution, the Lödige mixing speed was reduced to 79 rpm, and mixing was continued for 30 minutes. The cosolvents used in this example were: propylene glycol (PG), 1,3-propanediol (PDO), isopropyl alcohol (IPA), methanol (MeOH), and ethylene glycol (EG). The results can be seen in Tables 25-27.

TABLE 25

| | LUPAMIN | | | | Residence |
|---|---|---|---|---|---|
| | 9095 | Cosolvent | H₂O | Coating | Time |
| Example | (wt %) | (wt %) | (wt %) | Temp (° C.) | (min.) |
| 9a | 4.0 | 1% PG | 1.5 | 60 | 30 |
| 9b | 4.0 | 1% PDO | 1.5 | 60 | 30 |
| 9c | 4.0 | 1% IPA | 1.5 | 60 | 30 |
| 9d | 4.0 | 1% MeOH | 1.5 | 60 | 30 |
| 9e | 4.0 | 1% EG | 1.5 | 60 | 30 |

TABLE 26

| | | | | | | Gel | |
|---|---|---|---|---|---|---|---|
| | | | AUL | | | integrity | |
| | PSD | CRC | 0.9 psi | FS GBP | GBP 0.3 psi | (1~4 | |
| Example | (>850μ wt %) | (g/g) | (g/g) | (Darcies) | (Darcies) | scale) | SAP Surface |
| 9a | 0.22 | 24.59 | 16.90 | 342.5 | 2.45 | 4.0 | Hydrophobic |
| 9b | 0.23 | 24.65 | 16.39 | 286.5 | 3.76 | 4.0 | Hydrophobic |
| 9c | 0.38 | 23.30 | 16.62 | 289.8 | 3.53 | 4.0 | Hydrophobic |
| 9d | 0.33 | 24.55 | 17.50 | 302.6 | 3.83 | 4.0 | Hydrophobic |
| 9e | 0.70 | 24.64 | 16.60 | 328.9 | 3.00 | 4.0 | Hydrophobic |

TABLE 27
Wicking index (cm)

| Example | 1 min. | 5 min. | 10 min. |
|---|---|---|---|
| 9a | 0.4 | 1.0 | 1.5 |
| 9b | 0.8 | 1.0 | 2.0 |
| 9c | 0.5 | 1.5 | 2.5 |
| 9d | 1.8 | 2.5 | 3.0 |
| 9e | 0.5 | 1.4 | 2.0 |

### Example 10

Surface-crosslinked polymer particles, HySorb B-8700AD, were preheated in a laboratory oven set at a predetermined temperature. When the polymer particles (1 kg) attained the predetermined temperature, the particles were transferred to a preheated laboratory Lödige mixer. The polymer particles were maintained at the constant predetermined temperature throughout the coating step. Addition of a polyvinylamine coating solution (20 grams LUPAMIN 9095, 10 grams PG, and 15 grams DI water) to the preheated polymer particles performed dropwise over 5 minutes at a Lödige mixing speed of 449 rpm. After complete addition of the coating solution, the Lödige mixing speed was reduced to 79 rpm, and mixing was continued for 30 minutes. The results can be seen in Tables 28-30.

TABLE 28

| | | Propylene | | | |
|---|---|---|---|---|---|
| | LUPAMIN | glycol | | | Residence |
| | 9095 | (PG) | H₂O | Coating | Time |
| Example | (wt %) | (wt %) | (wt %) | Temp (° C.) | (min.) |
| 10a | 2.0 | 1.0 | 1.5 | 60 | 30 |
| 10b | 2.0 | 1.0 | 1.5 | 80 | 30 |
| 10c | 2.0 | 1.0 | 1.5 | 100 | 30 |
| 10d | 2.0 | 1.0 | 1.5 | 120 | 30 |
| 10e | 2.0 | 1.0 | 1.5 | 140 | 30 |

TABLE 29

| | | | | | | Gel | |
|---|---|---|---|---|---|---|---|
| | | | AUL | | | integrity | |
| | PSD | CRC | 0.9 psi | FS GBP | GBP 0.3 psi | (1~4 | |
| Example | (>850μ wt %) | (g/g) | (g/g) | (Darcies) | (Darcies) | scale) | SAP Surface |
| 10a | 0.19 | 25.43 | 18.57 | 307.4 | 6.83 | 3.5 | Hydrophobic |
| 10b | 0.18 | 25.64 | 18.82 | 297.8 | 7.95 | 4.0 | Hydrophobic |
| 10c | 0.05 | 26.03 | 19.30 | 284.7 | 7.56 | 4+ | Hydrophilic |
| 10d | 0.05 | 26.25 | 20.20 | 188.0 | 7.12 | 4+ | Hydrophilic |
| 10e | 0.04 | 26.40 | 20.50 | 142.2 | 7.43 | 4+ | Hydrophilic |

TABLE 30
Wicking index (cm)

| Example | 1 min. | 5 min. | 10 min. |
|---|---|---|---|
| 10a | 0.5 | 1.6 | 2.8 |
| 10b | 1.0 | 4.2 | 5.5 |
| 10c | 3.5 | 5.0 | 8.0 |
| 10d | 3.0 | 5.0 | 6.5 |
| 10e | 5.8 | 8.0 | 9.0 |

### Example 11

### Delayed Absorption Time

Polyvinylamine-coated polymer particles of Example 1 (1 g), having a hydrophilic surface, were transferred into a plastic weighing pan. Then, a colored 0.9% saline solution (1.0 g) was added onto the polymer particles by a disposable pipette. The time (in seconds) of saline solution disappearing from polymer particle surfaces was measured (Example 11 a). This procedure was duplicated for (a) surface-crosslinked HySorb B-8700AD particles that lacked a polyamine coating (Control), and (b) for polyamine-coated HySorb B-8700 particles of Example 2 having a hydrophobic surface (Example 11b). The results can be seen in Tables 31-33. FIG. 15 and the Tables 31-33 show the delayed absorption time phenomenon demonstrated by the present polyamine-coated SAP particles having a hydrophobic surface.

TABLE 31

| | | Propylene | | | |
|---|---|---|---|---|---|
| | LUPAMIN | Glycol | | | Residence |
| | 9095 | (PG) | H₂O | Coating | Time |
| Sample | (wt %) | (wt %) | (wt %) | Temp(° C.) | (min.) |
| Control | 0.0 | 0.0 | 0.0 | — | — |
| 11a | 4.0 | 0.0 | 1.5 | 60 | 30 |
| 11b | 4.0 | 1.0 | 1.5 | 60 | 30 |

TABLE 32

| | PSD | | AUL | | GBP | Gel |
|---|---|---|---|---|---|---|
| | (>850μ | CRC | 0.9 psi | FS GBP | 0.3 psi | Integrity |
| Sample | wt %) | (g/g) | (g/g) | (Darcies) | (Darcies) | (1–4 scale) |
| Control | | 26.28 | 19.60 | 85.5 | 8.48 | 1.0 |
| 11a | 5.94 | 24.90 | 17.23 | 229.0 | 6.59 | 4.0 |
| 11b | 0.22 | 24.59 | 17.45 | 342.5 | 2.45 | 4.0 |

TABLE 33

| | | Total Saline Disappear Time (sec) | | | |
|---|---|---|---|---|---|
| Sample | SAP Surface | Run 1 | Run 2 | Run 3 | Ave. |
| Control | Hydrophilic | 0.0 | 0.0 | 0.0 | 0.0 |
| 11a | Hydrophilic | 0.0 | 0.0 | 0.0 | 0.0 |
| 11b | Hydrophobic | 21.2 | 30.0 | 33.3 | 28.2 |

Typically, SAP particles have a hydrophilic surface, and absorb saline solution immediately upon contact between the solution the particles surface. Therefore, in contrast to the present SAP particles having a hydrophobic surface, conventional surface-crosslinked SAPs do not exhibit a time delay of liquid absorption.

In comparison to Example 1, Example 2 and Examples 9-11 show that polyamine-coated SAP particles of the present invention demonstrate excellent permeability (FSGBP), absorbency is maintained (CRC), and gel integrity (GI) is improved, in addition to a reduced agglomeration of particles and delayed swelling upon insult when the SAP particle surface is rendered hydrophobic by incorporating a cosolvent in the coating process.

The balanced properties of absorbency, permeability, and gel integrity demonstrated by the these examples, and the delayed swelling of the particles after insult, also are attributed to the relatively low temperature at which the surface-crosslinked SAP particles are maintained after application of the polyamine to the surfaces of the surface-crosslinked SAP particles. In particular, the low curing temperatures maintain an excellent gel integrity, which is adversely affected by a high temperature cure of the polyamine coating.

### Example 12

Handsheets were prepared using standard airforming handsheet equipment to yield a 10 inch by 17 inch (25.4 cm×43.2 cm) composite handsheet. A total of 37.13 grams of the SAP particles of several Examples above (shown in Table 34) and 19.99 grams of NB480 fluff fiber (both SAP and fiber weights include 5% waste due to accumulation near the walls of the airforming handsheet equipment) were used to create each example with a target basis weight of 496 grams per square meter (GSM). Forming tissue with a basis weight of 16.6 gsm (White Wrap Sheet, available from Cellu Tissue Holdings, Inc., a business having offices located in East Hartford, Conn., U.S.A.) was used for the top and bottom of the samples. A sheet of the forming tissue was placed on the bottom of the former.

The SAP of the Examples above and the NB480 were each divided into equal portions (6 portions of fluff and 5 portions of superabsorbent materials). Each fluff portion was then introduced into the top of the former, followed immediately with a superabsorbent portion, allowing the vacuum to disperse the fluff and superabsorbent materials into the former chamber and onto the forming tissue. This process was continued until the last portion of fluff was consumed, forming a substantially uniform distribution of fluff and superabsorbent materials.

A comparative example was also made by substituting HYSORB B-8700 AD (a conventional superabsorbent material available from BASF) for the SAP particles of the present invention. Another sheet of forming tissue was then placed on top of the sample, and the sample was placed into a CARVER PRESS model #4531 (available from Carver, Inc., a business having offices located in Wabash, Ind. U.S.A.) and densified to approximately 0.35 g/cc. Samples were then cut from the composite handsheets in appropriate sizes for testing, as set forth in the test procedures described above. The samples were then tested for Saturated Capacity (SAT CAP) and Intake Rate as described in the test procedures above. The results can be seen in Table 34.

TABLE 34

| | | | | | | 0.5 psi | 2^{nd} insult | 3rd insult |
|---|---|---|---|---|---|---|---|---|
| | | | Fiber | Basis | Composite | saturated | intake | intake |
| SAP | Fiber | SAP wt | wt | wt | density | capacity | rate | rate |
| type | type | (gm) | (gm) | (GSM) | (gm/cc) | (g/g) | (ml/sec) | (ml/sec) |
| Control | NB480 | 37.13 | 19.99 | 496 | 0.35 | 20.0 | 1.19 | 0.79 |
| Hysorb | | | | | | | | |
| B- | | | | | | | | |
| 8700AD | | | | | | | | |
| Example | NB480 | 37.13 | 19.99 | 496 | 0.35 | 20.7 | 1.51 | 1.08 |
| 3b | | | | | | | | |
| Example | NB480 | 37.13 | 19.99 | 496 | 0.35 | 20.4 | 1.49 | 1.14 |
| 3e | | | | | | | | |
| Example | NB480 | 37.13 | 19.99 | 496 | 0.35 | 20.8 | 1.52 | 1.09 |
| 7a | | | | | | | | |
| Example | NB480 | 37.13 | 19.99 | 496 | 0.35 | 21.1 | 1.73 | 1.24 |
| 10a | | | | | | | | |
| Example | NB480 | 37.13 | 19.99 | 496 | 0.35 | 21.7 | 1.47 | 0.97 |
| 10c | | | | | | | | |

From Table 34, it can be seen that absorbent structures comprising various SAP particles of the present invention generally exhibit an improved 2^{nd} and 3^{rd} Insult Intake Rate at a similar or improved Saturated Capacity when compared to conventional superabsorbent materials.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the desirable embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. An absorbent article comprising:
a topsheet;
a backsheet; and
an absorbent core disposed between the topsheet and the backsheet;
wherein the absorbent core includes superabsorbent polymer particles having a centrifuge retention capacity of at least about 25 g/g, a free swell gel bed permeability of at least 200 Darcies, and a gel integrity of at least 2.

2. The absorbent article of claim 1 wherein at least one of the topsheet, backsheet, and absorbent core is stretchable.

3. The absorbent article of claim 1 wherein the absorbent core comprises at least about 30% by weight of the coated superabsorbent polymer particles.

4. The absorbent article of claim 1 wherein the absorbent core comprises about 60% to about 95% by weight of the coated superabsorbent polymer particles.

5. The absorbent article of claim 1 wherein the absorbent core further comprises fluff.

6. The absorbent article of claim 1 wherein the absorbent core further comprises a surfactant.

7. The absorbent article of claim 1 wherein the absorbent core comprises layers.

8. The absorbent article of claim 7 wherein at least one of the layers comprises substantially the superabsorbent material and at least one of the layers comprises substantially fluff.

9. The absorbent article of claim 1 wherein the article is selected from personal care absorbent articles, health/medical absorbent articles, household/industrial absorbent articles and sports/construction absorbent articles.

10. The absorbent article of claim 1 wherein the superabsorbent polymer particles have a free swell gel bed permeability of at least 250 Darcies.

11. The absorbent article of claim 1 wherein the superabsorbent polymer particles have a gel integrity of at least 2.5.

12. The absorbent article of claim 1 wherein the surfaces of the superabsorbent polymer particles are hydrophobic.

13. The absorbent article of claim 1 wherein the surfaces of the superabsorbent polymer particles are hydrophilic.

14. The absorbent article of claim 13 wherein the superabsorbent polymer particles have been prepared by a method wherein particles of a surface-crosslinked superabsorbent polymer are coated with a coating composition comprising a polyamine and water; and
maintaining the polyamine-coated polymer particles at 25° C. to 100° C. for about 5 to about 60 minutes.

15. The absorbent article of claim 14 wherein the superabsorbent polymer particles have a hydrophilic surface.

16. The absorbent article of claim 14 wherein the surface-crosslinked superabsorbent polymer comprises acrylic acid, methacrylic acid, or a mixture thereof.

17. The absorbent article of claim 14 wherein the surface-crosslinked superabsorbent polymer has a degree of neutralization of about 25 to about 100.

18. The absorbent article of claim 14 wherein the polyamine is present on surfaces of the surface-crosslinked superabsorbent polymer in an amount of about 0.1% to about 2%, by weight, of the particle.

19. The absorbent article of claim 14 wherein the polyamine has one or more of primary amino groups, secondary amino groups, tertiary amino groups, and quaternary ammonium groups.

20. The absorbent article of claim 14 wherein the polyamine has a weight average molecular weight of about 5,000 to about 1,000,000.

21. The absorbent article of claim 14 wherein the polyamine is a homopolymer or a copolymer selected from a polyvinylamine, a polyethyleneimine, a polyallylamine, a polyalkyleneamine, a polyazetidine, a polyvinylguanidine, a poly(DADMAC), a cationic polyacrylamide, a polyamine functionalized polyacrylate, or mixtures thereof.

22. The absorbent article of claim 14 wherein the surface-crosslinked superabsorbent polymer further comprises a crosslinking agent.

23. The absorbent article of claim 22 wherein the crosslinking agent comprises a salt having (a) a polyvalent metal cation of valence +2, +3, or +4, (b) a polyvalent anion of valence −2, −3, or −4, or (c) a polyvalent cation and a polyvalent anion.

24. The absorbent article of claim 23 wherein the polyvalent metal cation is selected from the group consisting of Mg²⁺, Ca²⁺, Al³⁺,Sc³⁺, Ti⁴⁺, Mn²⁺, Fe^{2+/3+}, CO²⁺, Ni²⁺, Cu^{+/2+}, Zn²⁺, y³⁺, Zr⁴⁺, La³⁺, Ce⁴⁺, Hf⁴⁺, Au³⁺, an

25. The absorbent article of claim 23 wherein the polyvalent anion is selected from the group consisting of sulfate, phosphate, hydrogen phosphate, borate, an anion of a polycarboxylic acid, and mixtures thereof.

26. The absorbent article of claim 22 wherein the crosslinking agent comprises a multifunctional organic component capable of reacting with amino groups of the polyamine.

27. The absorbent article of claim 22 wherein the crosslinking agent is selected from an alkylene carbonate, a polyaziridine, a haloepoxy, a polyisocyanate, a di or polyglycidyl compound, a alkoxysilyl compound, urea, thiourea, guanidine, dicyandiamide, 2-oxazolidinone or a derivative thereof, bisoxazoline, a polyoxazoline, di and polyisocyanate, di and poly-N-methylol compounds, or a compound having two or more blocked isocyanate groups, a multifunctional aldehyde, a multifunctional ketone, a multifunctional acetal, a multifunctional ketal, or mixtures thereof.

28. The absorbent article of claim 14 wherein the surface-crosslinked superabsorbent polymer coating composition further comprises a cosolvent.

29. The absorbent article of claim 28 and the superabsorbent polymer particles have a hydrophobic surface.

30. The absorbent article of claim 28 wherein the cosolvent comprises an alcohol, a diol, a triol, or a mixture thereof.

31. The absorbent article of claim 28 wherein the cosolvent comprises methanol, ethanol, propyl alcohol, isopropyl alcohol, ethylene glycol, propylene glycol, an oligomer of ethylene glycol, an oligomer of propylene glycol, glycerin, a monoalkyl ether of propylene glycol, or mixtures thereof.

32. The absorbent article of claim 14 wherein the surface-crosslinked superabsorbent polymer comprises a surface-crosslinked polyacrylic acid.

33. The absorbent article of claim 32 wherein the polyamine comprises a polyvinylamine homopolymer or copolymer.

34. An absorbent article comprising:
a topsheet;
a backsheet; and
an absorbent core disposed between the topsheet and the backsheet;
wherein the absorbent core includes superabsorbent polymer particles have been prepared by:
(a) providing surface-crosslinked superabsorbent polymer particles;
(b) applying a composition comprising a polyamine, an optional cosolvent, and an optional crosslinking agent to surfaces of the surface-crosslinked polymer particles;
(c) maintaining the coated surface-crosslinked polymer particles of step (b) at about 25° C. to about 100° C. for a sufficient time to provide a cured polyamine coating on the surface-crosslinked polymer particles.

35. The absorbent article of claim 34 wherein maintaining step (c) is performed at about 50° C. to about 100° C. for about 5 minutes to about 60 minutes.

36. An absorbent article comprising:
a topsheet;
a backsheet; and
an absorbent core disposed between the topsheet and the backsheet;
wherein the absorbent core includes polyamine-coated superabsorbent polymer particles having a hydrophobic surface; and
wherein the polyamine-coated superabsorbent polymer particles have a wicking index of less than 2.3 centimeters after one minute, a free swell gel bed permeability at least two times greater than identical superabsorbent polymer particles free of a polyamine coating, and a gel integrity of at least 2.5.

37. The absorbent article of claim 36 wherein at least one of the topsheet, backsheet, and absorbent core is stretchable.

38. The absorbent article of claim 36 wherein the absorbent core comprises at least about 30% by weight of the coated superabsorbent polymer particles.

39. The absorbent article of claim 36 wherein the absorbent core comprises about 60% to about 95% by weight of the coated superabsorbent polymer particles.

40. The absorbent article of claim 36 wherein the absorbent core further comprises fluff.

41. The absorbent article of claim 36 wherein the absorbent core further comprises a surfactant.

42. The absorbent article of claim 36 wherein the absorbent core comprises layers.

43. The absorbent article of claim 42 wherein at least one of the layers comprises substantially the superabsorbent material and at least one of the layers comprises substantially fluff.

44. The absorbent article of claim 36 wherein the article is selected from personal care absorbent articles, health/medical absorbent articles, household/industrial absorbent articles and sports/construction absorbent articles.

45. The absorbent article of claim 36 wherein the polyamine-coated superabsorbent polymer particles have a wicking index of less than 3 centimeters after 5 minutes.

46. The absorbent article of claim 36 wherein the polyamine-coated superabsorbent polymer particles have a wicking index of less than 6.5 centimeters after 10 minutes.

47. The absorbent article of claim 36 wherein the polyamine-coated superabsorbent polymer particles have a gel integrity of at least 3.0.

48. The absorbent article of claim 36 wherein the polyamine-coated superabsorbent polymer particles have a gel integrity of at least 3.5.

49. The absorbent article of claim 36 wherein the polyamine-coated superabsorbent polymer particles, after contact with a 0.9% saline solution, exhibit a delay of at least five seconds prior to absorbing the saline solution.

50. The absorbent article of claim 36 wherein the polyamine-coated superabsorbent polymer particles have been prepared by a method wherein particles of a surface-crosslinked superabsorbent polymer are coated with a coating composition comprising a polyamine, a cosolvent, and water; and maintaining the polyamine-coated polymer particles at about 25° C. to about 100° C. for about 5 to about 60 minutes.

51. The absorbent article of claim 50 wherein the crosslinked superabsorbent polymer comprises acrylic acid, methacrylic acid, or a mixture thereof.

52. The absorbent article of claim 50 wherein the crosslinked superabsorbent polymer has a degree of neutralization of about 25 to about 100.

53. The absorbent article of claim 50 wherein the polyamine is present on surfaces of the surface-crosslinked superabsorbent polymer in an amount of about 0.1% to about 2%, by weight, of the particle.

54. The absorbent article of claim 50 wherein the polyamine has one or more of primary amino groups, secondary amino groups, tertiary amino groups, and quaternary ammonium groups.

55. The absorbent article of claim 50 wherein the polyamine has a weight average molecular weight of about 5,000 to about 1,000,000.

56. The absorbent article of claim 50 wherein the polyamine is a homopolymer or a copolymer selected from a polyvinylamine, a polyethyleneimine, a polyallylamine, a polyalkyleneamine, a polyazetidine, a polyvinylguanidine, a poly(DADMAC), a cationic polyacrylamide, a polyamine functionalized polyacrylate, or mixtures thereof.

57. The absorbent article of claim 50 wherein the cosolvent comprises an alcohol, a diol, a triol, or a mixture thereof.

58. The absorbent article of claim 57 wherein the cosolvent comprises methanol, ethanol, propyl alcohol, isopropyl alcohol, ethylene glycol, propylene glycol, an oligomer of ethylene glycol, an oligomer of propylene glycol, glycerin, a monoalkyl ether of propylene glycol, or mixtures thereof.

59. The absorbent article of claim 50 wherein the surface-crosslinked superabsorbent polymer comprises a surface-crosslinked polyacrylic acid.

60. The absorbent article of claim 50 wherein the polyamine comprises a polyvinylamine homopolymer or copolymer.

61. An absorbent article comprising:
a topsheet;
a backsheet; and
an absorbent core disposed between the topsheet and the backsheet;
wherein the absorbent core includes superabsorbent polymer particles have been prepared by:
(a) providing surface-crosslinked superabsorbent polymer particles;
(b) applying a composition comprising a polyamine and a cosolvent to surfaces of the surface-crosslinked polymer particles;
(c) maintaining the coated surface-crosslinked polymer particles of step (b) at about 25° C. to about 100° C. for a sufficient time to provide a cured, hydrophobic polyamine coating on the surface-crosslinked polymer particles.

62. The absorbent article of claim 61 wherein maintaining step (c) is performed at about 50° C. to about 100° C. for about 5 minutes to about 60 minutes.

63. The absorbent article of claim 62 wherein step (b) and step (c) are performed simultaneously.
